(19) United States
(12) Patent Application Publication
(10) Pub. No.: US 2018/0094209 A1
(43) Pub. Date: Apr. 5, 2018

(54) Extraction of Essential Oils

(71) Applicant: Sustainable Aquatics, Inc., Jefferson City, TN (US)

(72) Inventors: John Carberry, Talbott, TN (US); Matthew John Carberry, Talbott, TN (US)

(21) Appl. No.: 15/728,987

(22) Filed: Oct. 10, 2017

Related U.S. Application Data
(63) Continuation-in-part of application No. 14/847,829, filed on Sep. 8, 2015.
(60) Provisional application No. 62/050,318, filed on Sep. 15, 2014.

(51) Int. Cl. C11B 9/02; C12P 23/00; A23D 9/04

(52) U.S. Cl. CPC C11B 9/025; A23D 9/04; C12P 23/00

(57) ABSTRACT

Essential oils are extracted from a biomass through milling in a solvent to form a solution of the essential oil in the solvent. The solvent is or is part of a cover than reduces oxidative and other degradation of the essential oil during milling and isolation. The solubilized essential oil may be allowed to adhere to the originating milled biomass to form a feed or nutritional supplement. The solvent may be evaporated from the solubilized essential oil to form an essential oil concentrate. This essential oil concentrate may be used directly, adhered to a different biomass than the originating biomass, or used in combination with pharmaceutical, nutritional, or feed preparations. The essential oil concentrate is preferably adhered to the different biomass through milling under a cover to reduce oxidative and other degradation. The essential oil may be astaxanthin, capsaicin compounds, or cannabinoids.

### CROSS-REFERENCE TO RELATED APPLICATIONS

This application is a continuation-in-part of U.S. patent application Ser. No. 14/847,829, filed on Sep. 8, 2015, which claims the benefit of U.S. Provisional Patent Application Ser. No. 62/050,318, filed on Sep. 15, 2014, each of which is incorporated herein in its entirety by reference. This application further claims the benefit of U.S. Provisional Patent Application Ser. No. 64/406,226, filed on Oct. 10, 2016, incorporated herein in its entirety by reference.

### BACKGROUND

Essential oils include a wide range of oleoresins and other lipophilic, but somewhat polar, substances found in plants, algae, animal matter, and in some organic chemicals. Essential oils are of value in food manufacture, pharmaceuticals, nutraceuticals, animal feeds, cosmetics, spices, and chemicals.

Essential oils that are lipophilic, but with some polar character include the capsaicin and dihydrocapsaicin molecules from the fruit of habanero peppers. These compounds are considered oleoresin carotenoids. In addition to the capsaicin and dihydrocapsaicin molecules, habanero peppers include other carotenoids and oleoresins of potential value. A molecular representation of capsaicin (C₁₈H₂₇NO₃) is provided below in Structure I, where the non-polar end of the molecule includes ethylene functionality and the polar end includes amide, ether, and alcohol functionality. Dihydrocapsaicin is the same molecule where the ethylene functionality is hydrogenated.

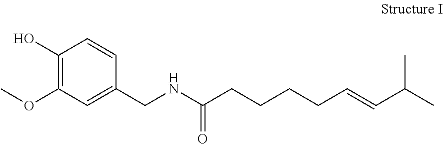 [SMILES: COc1cc(CNC(=O)CCCC/C=C/C(C)C)ccc1O]

Another example of an essential oil that is lipophilic, but with some polar character is astaxanthin, a keto-carotenoid, which is a phytochemical belonging to the class of molecules known as terpenes. A molecular representation of free astaxanthin (C₄₀H₅₂O₄) is provided below, where the non-polar central “backbone” separates terminal polar ester and alcohol functionality.

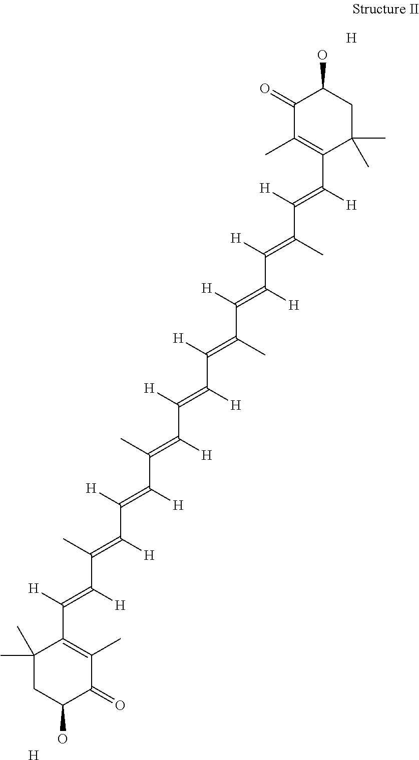

Astaxanthin is highly desired as a pharmaceutical and nutraceutical ingredient for human consumption and as a food and feed additive in agriculture and aquaculture. Astaxanthin provides the color and antioxidant functionality to several fish and animal meats, including salmon and egg yolks. Animals such as shrimp, krill, zooplankton, and salmon take up and display astaxanthin in their color, and astaxanthin contributes to the antioxidant value of their flesh or biomass when consumed by other animals. Astaxanthin also provides the red color of various other fish meats, such as trout and several cooked shellfish, such as shrimp and lobster.

Astaxanthin, similarly to other carotenoids, cannot be synthesized by animals and must be provided from the diet. Thus, mammals, including humans, lack the ability to synthesize astaxanthin. Lower animals, such as rotifers, as often grown to feed fish larvae in closed system aquaculture, also do not synthesize astaxanthin, thus producing fish larvae lacking the astaxanthin the fish larvae would normally obtain in nature through a copepod diet.

The essential oil astaxanthin occurs naturally in algae, bacteria, and yeasts. Haematococcus pluvialis, a fresh water alga, is the most productive source presently known for obtaining natural astaxanthin. Astaxanthin concentrations in Haematococcus pluvialis are known to exceed 40,000 parts per million.

The concentration of astaxanthin within the Haematococcus pluvialis algae cells is significantly heightened when the algae form astaxanthin-rich cysts. The vegetative, flagellated Haematococcus pluvialis algae produce cysts, a dormant or resting state of the algae, when subjected to stress inducing unfavorable temperatures, lack of sufficient light, and lack of sufficient nutrients. This dormant state can last for decades during which the cyst form of the algae can be dried, dehydrated, and eaten by animals. When the stress is reduced and growth conditions become more favorable, the dormant cysts of the algae can germinate into vegetative algae cells. The algae are believed to store the astaxanthin within the cysts to protect the cysts against oxidative damage until stress reduction and re-germination into vegetative algae cells occurs. As the cysts are substantially indigestible and resistant to digestive acids and enzymes, animals eating the stressed algae cysts can potentially transport the cysts to a location having more favorable growth conditions.

Astaxanthin stored within Haematococcus pluvialis algae cysts has exceedingly low bioavailability due to the hardness and indigestibility of the cysts. However, even if the astaxanthin is released from the cysts into an aqueous environment, the astaxanthin forms dimers and other aggregates that reduce bioavailability. This aggregation is believed attributable to the overall polarity of the astaxanthin molecule being low in relation to water, and due to the polarity of the astaxanthin molecule being isolated at the ends of the non-polar “middle” of the astaxanthin molecule.

While dormant cysts of Haematococcus pluvialis algae are relatively rich in astaxanthin, the astaxanthin is a small fraction by weight of the total algae/cyst biomass. For example, the astaxanthin containing carotenoid fraction of Haematococcus pluvialis algae typically constitutes approximately 2-7% of the dry body mass of the algae by weight. Of this 2-7% carotenoid fraction, approximately 70% is monoesters of astaxanthin, approximately 10% is diesters of astaxanthin, and approximately 5% is free astaxanthin by weight. The remaining approximately 15% of the 2-7% carotenoid fraction is typically a mixture of beta-carotene, canthaxanthin, lutein, and other compounds.

In addition to natural sources, such as Haematococcus pluvialis algae cysts, astaxanthin also is available from several synthetic sources. However, conventional synthetic production results in different ratios of the three astaxanthin stereoisomers in relation to the naturally produced stereoisomer ratios. The synthetic stereoisomer ratio of approximately 1:2:1 (3S,3′S:3R,3′S:3R,3′R) fails to produce the same color in feeds as the naturally derived stereoisomer ratio, which favors the SS stereoisomer.

Current processes for natural astaxanthin production via controlled growth of algae typically involve setting up a growth phase of algae, often in ponds or bioreactors filled with water. Such bioreactors can be indoors using artificial light sources or outdoors using sunlight. During this stage of production, typically 8 to 10 days or longer, nutrition is added to the water including the Haematococcus pluvialis algae culture. Nutritional elements may include nitrates, phosphates, sodium, and silicates, as needed to facilitate algal growth.

The grown algae are then subjected to a stress phase to promote the production of cysts, and thus astaxanthin, by the algae. Typically, stress is accomplished by subjecting the algae to nutritional withdrawal in conditions otherwise optimal for photosynthesis, i.e., in the presence of sufficient moisture, warmth, light, and carbon dioxide, and absent competition from other species. Thus, the stress phase relies on the algae to consume the nutrients in the water to depletion or near depletion before significant cyst formation. However, this “stress through starvation regiment” is a relatively long process that leaves many of the cells in a state resulting in death, not cyst formation. This death and subsequent decay of a portion of the algae cells may result in undesirably low yields of astaxanthin as a percentage of total algal biomass and further result in undesired contaminants in the algae and water mixture. While astaxanthin should theoretically approach or exceed 4% by weight of the biomass after the stress phase, the obtained astaxanthin concentration is often much lower due to death of algae cells containing relatively low concentrations of astaxanthin. The goal of the growth phase is to stress grown algal cells to produce astaxanthin-rich cysts, not to kill the grown cells before they can produce cysts.

In addition to the difficulties of algae/cyst production, many of the conventional processing techniques for breaking down the cell walls of the Haematococcus pluvialis cysts are difficult, cumbersome, and/or destructive to the astaxanthin molecule. The relatively high density and hardness of the astaxanthin containing cysts makes the cysts largely indigestible if consumed by animals, thereby limiting the bioavailability of the astaxanthin contained within the cysts. Conventional harvesting processes to free the astaxanthin from the astaxanthin-rich cysts typically include three stages. First, a mixture of water and algae with the included cysts is centrifuged to remove water. Then, the dehydrated algae is ground and/or treated with acid in an attempt to break the algae cells and cysts to liberate the astaxanthin. While acids are capable of breaking the cell walls of Haematococcus pluvialis cells and the cysts, the acids can also oxidatively or otherwise degrade the astaxanthin released from the cells, especially if not carefully exposure time and pH controlled. Conventional methods for grinding cyst-enriched Haematococcus pluvialis cells tend to be imprecise and can result in the oxidation of the astaxanthin molecule. Neither can they grind to a small enough particulate size to have a substantial impact on the cysts. Too much thermochemical stress through the use of heat or heat generated during grinding to break the cysts also can oxidatively and otherwise degrade the astaxanthin. Finally, the mixture of broken algae cells, broken cysts, and liberated astaxanthin is spray dried or otherwise prepared for packaging. However, the bioavailability of astaxanthin extracted by these conventional methods is very low, generally below 15% of the weight of the essential oil in the originating biomass is extracted.

Other examples of essential oils that are lipophilic, but with some polar character are the Tetrahydrocannabinol (THC) and Cannabidiol (CBD) oils present in the cannabis sativa plant. A molecular representation of cannabidiol (C₂₁H₃₀O₂) is provided below in Structure III, where the non-polar end of the molecule includes a non-polar five-carbon alkane chain connected to a polar alcoholic “middle” and then a relatively non-polar hexene/ethylene end. The cannabis plant includes other cannabinoids that are lipophilic, but with some polar character. Multiple varieties of the cannabis plant exist, some with approximately 0.3% or less cannabinoids by weight.

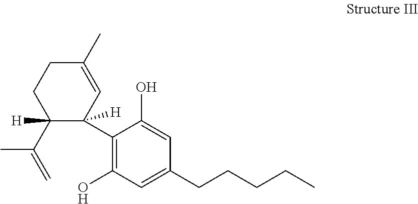 [SMILES: C=C(C)[C@@H]1CCC(C)=C[C@H]1c1c(O)cc(CCCCC)cc1O]

In addition to capsaicin compounds, cannabis compounds, and astaxanthin, other lipophilic essential oils having some polar character also are desirable for extraction and concentration. Some examples of animal-based essential oils include extracts from sea foods, such as green shell mussels, shark cartilage, shell fish, collagen extracts, docosahexaenoic acid (DHA), eicosapentaenoic acid (EPA) from fish, and lecithin from egg yolk. Some examples of additional plant-based essential oils include concentrated oils from peppers, such as jalapeno and others, concentrated oils from tobacco, garlic oil from garlic bulbs, lycopene from tomatoes, agar from agarwood, ajwain oil from the leaves of carum copticum, angelica root oil from angelica archangelica, anise oil from the pimpinella anisum, asafetida oil, balsam or peru from myroxylon, basil oil from basil, hop concentrate, germ oil from wheat, ginsenoside from ginseng, oil of grape seed, pigments from chili, and the like. The paper entitled Supercritical Fluid Extraction from Vegetable Materials, Helena Sovova, and Roumiana P. Stateva, Rev Chemical Engineering 27 (2011) by Walter de Gruyter, Berlin DOI 10.15.15/REVCE 2011.002, Table 1, p. 84 provides a list of essential oils and other materials that may be extracted.

To realize the enhanced nutritional or pharmacological value provided by essential oils when consumed as food or as a feed additive, the essential oils first require extraction from a biological source and processing into a relatively pure concentrate with acceptable bioavailability. Protecting the essential oil concentrate from oxidation also may be desired to provide acceptable bioavailability when consumed after storage. Optimally, the bioavailability of the extracted and concentrated essential oil would be 100%; however, such bioavailability performance is unlikely to be attained. Thus, an extracted and concentrated essential oil having a bioavailability of approximately 70% would be acceptable for most applications as only 30% of the “essential oil” is inactive. However, in the reverse instance where only 30% of the “essential oil” is bioavailable, the extracted and concentrated essential oil has little usefulness as approximately 70% of the material is inactive. Such low bioavailability for conventionally extracted essential oils means that the majority of the extracted and concentrated essential oil included in a feed, nutraceutical, pharmaceutical, or other final preparation is inactive. Thus, for commercial viability, extracted and concentrated essential oil production requires industrialization at economic scales to achieve low production costs, but a key factor is the bioavailability of the extracted essential oil.

In an attempt to ameliorate the disadvantages of conventional acid, milling, and heating techniques for essential oil extraction, supercritical carbon dioxide extraction (“SCCO2”) has also been used. SCCO2 methods attempt to extract a portion of the essential oil from the remaining cellular matter without resorting to acids and/or heat. However, SCCO2 methods use carbon dioxide, which has a low polarity and therefore poor ability to solubilize lipophilic essential oils—this is especially true for astaxanthin. To overcome the non-polarity of the carbon dioxide solvent, methanol or ethanol may be added to the carbon dioxide to increase solvent polarity. However, the amount of alcohol that may be added to the supercritical carbon dioxide is limited if the beneficial extraction abilities of the supercritical carbon dioxide is to be retained. Thus, the more polarity added to the supercritical extraction fluid with alcohols, the less supercritical extraction effect is retained.

Another issue with SCCO2 extraction in the astaxanthin/cyst context is cyst diminution during extraction. While the cysts have an original diameter of approximately 60 microns, the cysts contract to approximately 3-5 microns during extraction. This low temperature induced contraction is believed to damage the astaxanthin molecule in addition to the extraction process chiefly extracting 3-5 micron indigestible, contracted cysts instead of non-contracted indigestible 60 micron cysts or the bioavailable astaxanthin molecule. The SCCO2 extraction process is also believed to oxidatively degrade the astaxanthin that is successfully extracted. Thus, the bioavailability for SCCO2 extracted astaxanthin remains very low.

As can be seen from the above description, there is an ongoing need for improved processes for isolating essential oils that include improved methods and processes for producing essential-oil-enhanced biomass and improved methods and processes for extracting and concentrating the essential oils from the essential-oil-enhanced biomass. There also is a need for an improved process that provides a relatively high yield of purified, substantially non-oxidized essential oils and that precludes or reduces thermochemical stress, oxidation, and contamination of the essential oils during extraction and concentration.

### SUMMARY

In one aspect, a method of extracting an essential oil from a biomass includes combining a biomass including an essential oil with a cover within an attrition mill, where the essential oil is soluble in the cover and the attrition mill includes milling media; milling the biomass and the cover in the mill for a duration; reducing the particulate size of the biomass during the milling by repeatedly contacting the biomass with the milling media; releasing the essential oil from the biomass to the cover during the milling; dissolving at a portion of the essential oil released from the biomass in the cover during the milling, where the cover reduces the oxidation of the released essential oil in relation to the milling without the cover; forming a mixture during the milling including a solution of the essential oil in a solvent, where the essential oil is a solute and the cover includes the solvent, and a milled byproduct biomass; and separating the solution from the milled byproduct biomass, where the solution includes the essential oil.

In another aspect, a method of producing a solution of astaxanthin from a Haematococcus pluvialis algae biomass includes combining an initial feedstock of healthy algae and nutrients in water; amplifying the algae concentration in the water during a growth phase, the growth phase including supplying light from a light source and carbon dioxide to the initial feedstock, and supplying the nutrients in the water; removing at least a portion of the nutrients from the water after the growth phase; stressing the amplified algae by supplying additional light and carbon dioxide to the amplified algae to promote cyst formation by the amplified algae; combining the amplified algae with ethanol in a mill; milling the amplified algae in the ethanol in the mill to release astaxanthin to the ethanol; reducing oxidation of the astaxanthin with the ethanol; dissolving at least a portion of the astaxanthin in the ethanol to form a mixture including a solution of astaxanthin in the ethanol, where the astaxanthin is a solute and the ethanol is a solvent, and a milled byproduct of the amplified and stressed algae; and separating the solution from the milled byproduct of the amplified algae.

In another aspect, a method of manufacturing a bioavailable essential oil enriched food additive or feed includes combining a biomass including an essential oil with a first cover within a mill, where the essential oil is soluble in the first cover; milling the biomass and the first cover in the mill to release the essential oil to the first cover, where the first cover reduces oxidation of the essential oil, and at least a portion of the essential oil dissolves in the first cover to produce a mixture including a solution of the essential oil in the first cover, where the essential oil is a solute and the first cover includes a solvent, and a milled byproduct biomass; separating the solution from the milled byproduct biomass; blending the solution with an edible material; milling the solution and the edible material to transfer at least a portion of the essential oil from the solvent to the edible material; and removing the solvent from the edible material to provide the bioavailable essential oil enriched feed or food additive as the edible material including transferred essential oil.

In another aspect, a method of manufacturing a bioavailable essential oil enriched food additive or feed including biomass originating the essential oil includes combining a biomass including an essential oil with a cover within a mill, where the essential oil is soluble in the cover; milling the biomass and the cover in the mill to release the essential oil to the cover, where the cover reduces oxidation of the essential oil, and at least a portion of the essential oil dissolves in the cover to produce a mixture including a solution of the essential oil in the cover, where the essential oil is a solute and the cover includes a solvent, and a milled byproduct biomass; washing the milled byproduct biomass with additional solvent for the essential oil to provide additional solution including the essential oil; and separating the solution and the additional solution from the milled byproduct biomass to provide the bioavailable essential oil enriched food additive or feed as the milled byproduct biomass.

Other systems, methods, features and advantages of the invention will be, or will become, apparent to one with skill in the art upon examination of the following figures and description. It is intended that all such additional systems, methods, features, and advantages be included within this description, be within the scope of the invention, and be protected by the claims that follow. The scope of the present invention is defined solely by the appended claims and is not affected by the statements within this summary.

### BRIEF DESCRIPTION OF THE DRAWINGS

The figures represent example techniques and structures designed to carry out the objects of the present general inventive concept, but the present general inventive concept is not limited to these examples. In the accompanying drawings and illustrations, the sizes and relative sizes, shapes, and qualities of lines, entities, and regions may be exaggerated for clarity. A wide variety of additional techniques and structures will be more readily understood and appreciated through the following description, with reference to the accompanying drawings.

FIG. 1 represents a method of producing and extracting at least one essential oil from a biological source.

FIG. 2 is a cross-sectional side view representing a bioreactor useful in conducting a growth phase.

FIG. 3 is a schematic representation of a system that may be used to accomplish several operations of the method.

FIG. 4 is a cross-sectional side view illustrating a filter useful in performing the nutrient removal operation of the method.

FIG. 5 is another schematic representation of a system that may be used to accomplish several operations of the method.

FIG. 6 represents a method of extracting essential oils from an essential oil enriched biomass.

FIG. 7 represents a method of extracting essential oils from a biomass.

FIG. 8A represents a method of extracting essential oils from a biomass.

FIG. 8B is a graph showing the bioavailability of astaxanthin prepared using a conventional SCCO2 technique in comparison to the described methods.

FIG. 9 represents a method of manufacturing a gelatin-based vitamin supplement.

FIG. 10 represents a method of manufacturing a bioavailable essential oil enriched feed or food additive with a previously extracted essential oil.

FIG. 11 represents a method of manufacturing a bioavailable essential oil enriched feed or food additive including the biomass originating the essential oil.

### DETAILED DESCRIPTION

A method of enhancing, extracting, and concentrating essential oils from biological sources for potential use in food manufacture, pharmaceuticals, nutraceuticals, animal feeds, cosmetics, spices, chemical manufacture, and the like is described. The essential oils are extracted from a biomass through milling in a solvent to form a solution of the essential oil in the solvent. The solvent is or is part of an oxygen-excluding cover fluid than reduces oxidative and other degradation of the essential oil during milling and isolation. The solubilized essential oil may be allowed to adhere to the originating milled biomass to form a feed or nutritional supplement. The solvent may be evaporated from the solubilized essential oil to form a carotenoid concentrate. This carotenoid concentrate may be used directly, adhered to a different biomass than the originating biomass, or used in combination with pharmaceutical, nutritional, or feed preparations. The carotenoid concentrate is preferably adhered to the different biomass through milling under a cover to reduce oxidative and other degradation. The essential oil may be astaxanthin or capsaicin compounds.

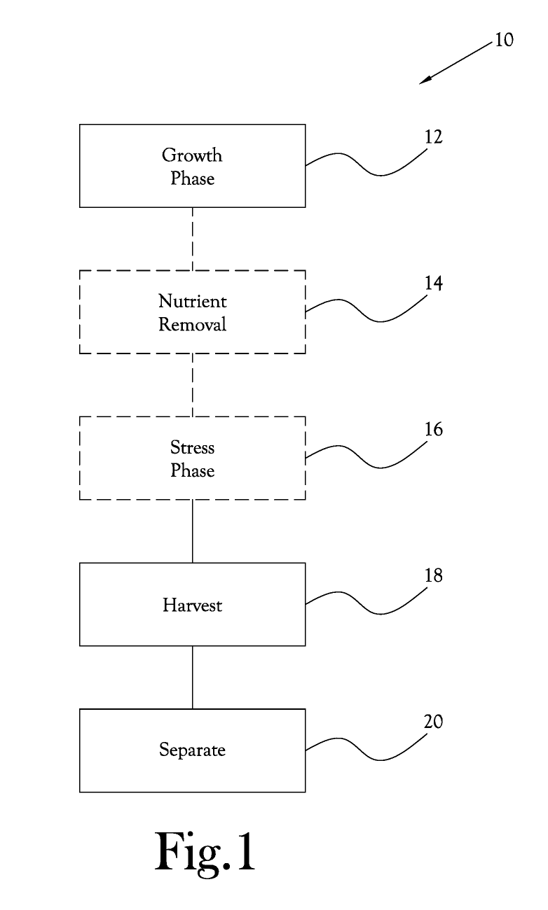

FIG. 1 represents a method 10 of producing and extracting at least one essential oil from a biological source. The essential oils include a wide range of oleoresins and other lipophilic, but somewhat polar, substances found in plants, algae, animal matter, and in some organic chemicals. The biological sources of essential oils include algae, plants, fungi, molds, and the like.

In growth phase 12, the biological source of the essential oil is grown. When the essential oil is capsaicin, for example, the habanero pepper plant may be grown in soil, hydroponically, or aquaponically, and the like. When the essential oil is THC or CBD, the cannabis plant may be similarly grown. Alternatively, when the essential oil is astaxanthin, the algae Haematococcus pluvialis may be grown in water containing nutrients under conditions conducive to growth.

For algae, in the growth phase 12, a mixture including water and an initial stock of algae is introduced to a bioreactor. One or more nutrients are added to the water before, after, or during the initial algal stock introduction to the bioreactor. The nutrients may include nitrates, phosphates, sodium, and silicates, and the like. Other nutrients and growth promotors may be added depending on the specific type of algae growth desired. The algae are then exposed to light and sufficient carbon dioxide to promote the desired growth. The mixture may be heated or cooled to a temperature desirable for the specific type of algae growth desired.

For algae that form essential oil rich cysts, optional nutrient removal 14 follows the growth phase 12. The optional nutrient removal 14 may be used for other plants that increase essential oil production when growth nutrients are reduced. In the nutrient removal 14, the growth nutrients may be rapidly reduced and/or removed from the algae. For Haematococcus pluvialis algae, for example, the nutrients in the water are reduced by at least 50% in relation to their concentration during the growth phase 12. Preferably, the nutrients in the water are reduced by at least 90%, more preferably by at least 92%, in relation to their concentration during the growth phase 12. While not shown in FIG. 1, in addition to, or in place of the nutrient removal 14, the salinity of the water may be significantly increased for algae. A significant increase is an at least 20% concentration increase (weight/weight) of salt in the water in relation to the salt concentration during the growth phase 12.

If the nutrient removal 14, salting, or other stress inducing technique is implemented to increase essential oil production, stress phase 16 follows. The Haematococcus pluvialis algae cells enter the stress phase 16, for example, where the essential oil astaxanthin is concentrated in cysts in response to the nutrient removal 14. Preferably, while the algae are stressed during the stress phase 16, the algae are not killed. Instead, in relation to unstressed algae in the growth phase 12, a relatively high yield of healthy, astaxanthin-enhanced Haematococcus pluvialis cysts are produced.

The stress phase 16 may result in the production of an amount of astaxanthin by Haematococcus pluvialis algae in excess of 1.5% of the dry weight of the algae. Preferably, the stress phase 16 may result in the production of an amount of astaxanthin by the Haematococcus pluvialis algae approaching, or approximately equal to 4% of the dry weight of the algae. The stress phase 16 may result in the production of an amount of astaxanthin by the Haematococcus pluvialis algae from 2-7% of the dry weight of the algae, with approximately 70% of the carotenoid fraction of the Haematococcus pluvialis algae being monoesters of astaxanthin, approximately 10% being diesters of astaxanthin, approximately 5% being free astaxanthin, and the remainder being a mixture of beta-carotene, canthaxanthin, lutein, and other substances.

In harvest phase 18, the essential oil including biomass of the biological source is harvested. In the case of pepper plants, the peppers may be collected. In the case of cannabis plants, the flowers, the leaves, or the stalks, and any combination thereof, may be collected. In the case of algae, harvesting may be conducted by filtration, centrifugation, and the like to remove the essential oil rich algae from the water, nutrient, and optional salt mixture. Regardless of the harvesting method used, a biomass with essential oil content and little residual water is produced. The biomass also may be dried prior to milling, as discussed further below.

In separation 20, the essential oils are separated from the harvested biomass to isolate essential oils from the biomass. Preferably, the separation 20 reduces the oxidation of the essential oils as the essential oils are separated and isolated from the biomass.

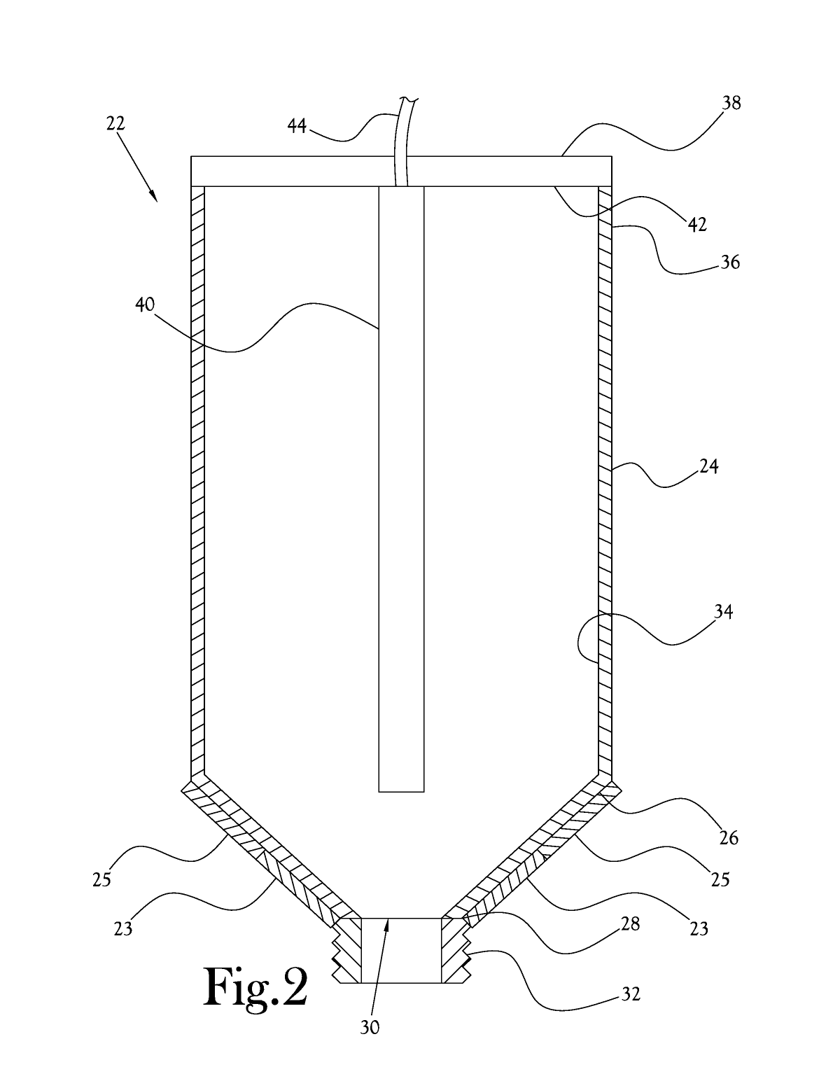

FIG. 2 is a cross-sectional, side view representing a bioreactor 22 useful in conducting a growth phase for algae, such as the growth phase 12 of FIG. 1. The bioreactor 22 includes a substantially elongate, cylindrical vessel 24, having a vertically-extending central axis and defining a frustoconical tapered portion 26 at a lower end. A lower end 28 of the tapered portion 26 defines an opening 30 in fluid communication with a coupler 32. The coupler 32 is configured to establish a substantially fluid tight connection with a pipe, hose, or other such conduit (not shown) providing fluid communication to another vessel. A valve (not shown) may be placed in the coupler 32, in the conduit, between the coupler 32 and the conduit, and the like to regulate material transfer from the bioreactor 22. The valve permits the user to close the lower end 28 of the tapered portion 26, thereby establishing a substantially material tight volume internal of the vessel 24 for holding water or other material. The valve may be adjusted between open and closed positions to selectively allow or disallow liquid to flow through the opening 30. Thus, liquid received within the vessel 24 may be removed from the vessel 24 by selectively opening the valve and allowing the liquid to drain from the vessel 24. Alternatively, the valve may be closed to configure the bioreactor 22 to hold liquid.

The bioreactor 22 also includes a closed or closable upper end 36. For example, a lid 38 is configured to mate with and close the upper end 36 of the vessel 24. A light source 40 may be configured along an interior surface 42 of the lid 38 and may be configured to extend into the interior of the vessel 24. The light source 40 may include an elongate fluorescent light mounted to the lid interior surface 42 and configured such that, when the lid 38 is mated with the upper end 36 of the vessel 24, the fluorescent light extends along a central axis of the bioreactor 22. In addition to fluorescent, LED, incandescent, HID, halide, and other light sources may be used that provide sufficient light intensity and temperature to promote algal growth.

Suitable wiring 44 and other hardware and software may be provided to supply electricity to power the light source 40 and to allow the light source 40 to be turned on and off. Thus, when the lid 38 is mated with the upper end 36 of the vessel 24, the light source 40 extends generally along a central axis of the bioreactor 22 and may be activated to provide light to the interior of the bioreactor 22.

The vessel 24 may be fabricated from any of a number of substantially rigid materials compatible with algal growth. Preferably, the vessel 24 is fabricated from one or more materials, at least one of which assists in confining light emanated from the light source 40 to an interior of the bioreactor 22. For example, the vessel 24 may be fabricated from an opaque material, such as metal, plastic, opaque fiberglass, or the like. Preferably, the vessel 24 is at least diffusely reflective of light, such that at least a portion of light from the light source 40 reaching the walls of the vessel 24 is reflected back into the vessel interior. For example, in FIG. 2, the vessel 24 may be fabricated from a fiberglass material having a layer of white gelcoat along an interior surface 34 thereof. The white gelcoat is diffusely reflective of light striking the interior surface 34 of the vessel 24 and the fiberglass material is substantially opaque. Thus, light from the light source 40 reaching the interior surface 34 of the vessel 24 is diffusely reflected back into the interior of the vessel 24. Alternatively, the interior surface 34 of the vessel 24 may define a mirrored surface finish configured to produce specular reflection of light striking the interior surface 34 of the vessel 24. Other materials and configurations may be used in the fabrication of the vessel 24 that enhance algal growth.

Preferably, a plurality of heating and cooling mechanisms are provided either within or proximate the bioreactor 22 and are configured to provide heat to and/or withdraw heat from the interior of the bioreactor 22. For example, a plurality of heating pads (not shown) may be provided along the exterior of the lower portion 26 of the bioreactor 22. The heating pads may be configured to provide and direct heat toward the bioreactor 22. Thus, the heating pads may be activated to selectively warm the contents of the bioreactor 22. Likewise, a plurality of cooling pads (not shown) may be provided along the exterior of the lower portion 26 of the bioreactor 22. The cooling pads may be configured to draw heat from the exterior surface of the bioreactor 22. Thus, the cooling pads may be activated to selectively cool the contents of the bioreactor 22. Other heating and cooling mechanisms and arrangements may be used to allow the contents of the bioreactor 22 to be selectively heated and cooled. For example, one or more heating and/or cooling coils of the type fabricated from thermally conductive materials may be provided within the interior of the bioreactor 22 and configured to transfer heat to and/or from the bioreactor interior.

Additional structures and devices may be used as a bioreactor to accomplish the growth phase 12 of FIG. 1. For example, a bioreactor may be in the form of a single-use transparent bag having a diameter of approximately 25-30 centimeters and a height of approximately two meters. Alternatively, one or more drums, tanks, containers, pools, ponds, or the like may be used to accomplish the initial setup of the growth phase 12.

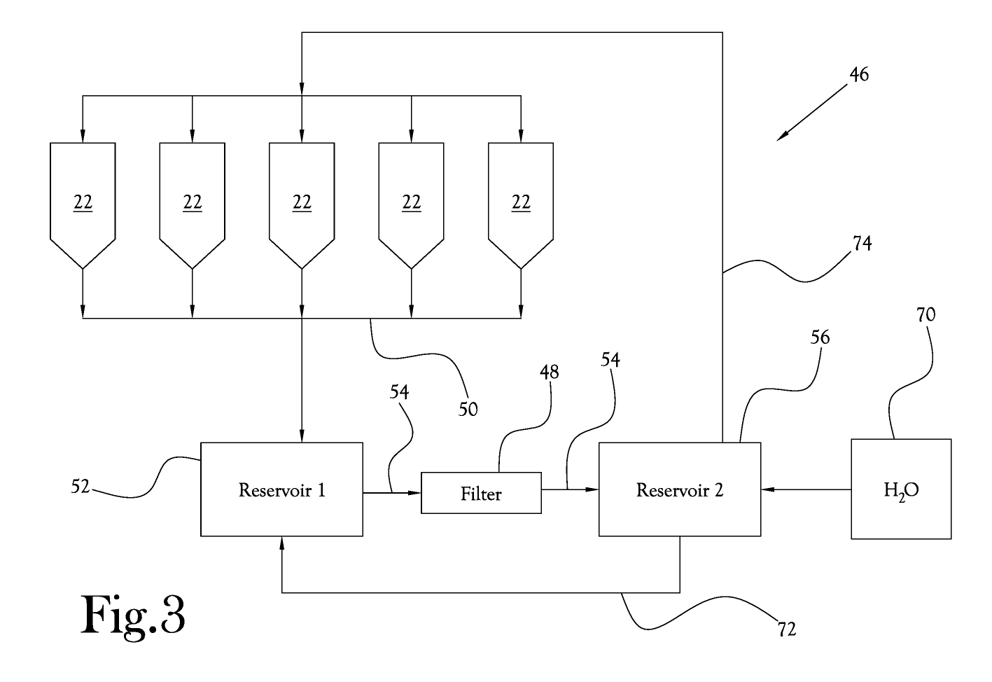

FIG. 3 is a schematic representation of a system 46 that may be used to accomplish several operations of the method 10 of FIG. 1. A plurality of bioreactors, such as the bioreactor 22 of FIG. 2 may be used. The plurality of bioreactors 22 may be loaded with an initial stock of algae and nutrients in water. The water mixture is exposed to an amount of light and carbon dioxide favorable for growth of the algae, and each mixture may be maintained at a temperature favorable for growth of the algae. In this manner, the bioreactors 22 are configured to allow and promote growth of algae within the bioreactor 22.

The initial mixture of water and algae cells is exposed to light via the light source 40 within the bioreactors 22. When the interior of the vessel 24 is reflective to light, light may be emitted in a 360-degree pattern outwardly from the light source 40 and reflects from the interior 34 of the vessel 24, such that the algae within each bioreactor 22 is exposed to light from a plurality of directions. If the vessel 24 is fabricated from a transparent or translucent material, one or more exterior light sources may be provided outside each bioreactor 22 and configured to direct light into the interior of each vessel 24.

Sufficient turbulence and/or agitation is maintained within the bioreactors 22 to allow a significant portion of the algae cells within the bioreactors 22 to have at least intermittent exposure to the light within the bioreactors 22, as well as carbon dioxide and nutrients. For example, carbon dioxide is supplied to the water and algae mixture within the bioreactors 22 in the form of gas flow from the lower portion 26 of the bioreactors 22 to the upper portion 36 of the bioreactors 22. More specifically, a mixture of carbon dioxide and air may be pumped, via an air pump and suitable conduit, into an interior of the lower portion 26 of the bioreactors 22. This carbon dioxide and air mixture is allowed to diffuse and rise to an upper surface of the water and algae mixture within the bioreactors 22, thereby providing carbon dioxide to promote growth of the algae within the bioreactors 22 and to stabilize the pH within the bioreactors 22. This upward gas flow further serves to gently agitate the water and algae mixture within the bioreactors 22 with minimal damage to the algae, such that the algae circulates within the bioreactors 22 to expose a significant portion of the algae to the nutrients within the water, while also allowing the algae to at least intermittently receive light from the light source 40 without being shaded by adjacent algae.

Other devices and configurations may be used to expose the algae to the light, carbon dioxide, and nutrients supplied within the bioreactors 22. For example, an impeller or other mechanical mixing device may be provided to stir or otherwise agitate the water and algae mixture within the bioreactors 22. However, such mixing devices should preferably be configured to result in minimal damage and/or degradation to the algae within the bioreactors 22.

After set-up, the bioreactors 22 are maintained within a temperature range and in conditions conducive to growth of algae for a period of time sufficient to allow growth of the algae to a desired algal density in the water. For example, the bioreactors 22 may be maintained at a temperature of from 20 to 36 degrees Celsius (approximately 68 to 96.8 degrees Fahrenheit) for a period from 8 to 12 days. Preferably, the bioreactors 22 are maintained at a temperature of from 22 to 34 degrees Celsius (approximately 71.6 to 93.2 degrees Fahrenheit), and more preferably from 25 to 28 degrees Celsius (approximately 77 to 82.4 degrees Fahrenheit), for a period of between approximately 8 to 12 days. The bioreactors 22 may be maintained at a temperature from 27 to 29 degrees Celsius (approximately 82.4 degrees Fahrenheit) for a period from 8 to 12 days.

After set-up, the bioreactors 22 may be maintained at a temperature from 21 to 23 degrees Celsius and at a pH of 7.2 to 7.8, preferably at a pH of 7.4 to 7.6. Throughout this time, additional nutrients are optionally added to the interior of the bioreactors 22 to replace any nutrients consumed by the algae growing therein, and to maintain a supply of suitable nutrients within the bioreactors 22 for further algal growth. To the extent water is lost from one or more bioreactors 22 due to evaporation or other losses, additional water is optionally added to maintain the desired amount of water and algae mixture within the bioreactors 22. Additional adjustments to the water and algae mixture may optionally be made, via water additives and the like to maintain suitable pH, water chemistry, and water quality within the bioreactors 22 as conducive to the desired algal growth.

The carbon dioxide and air mixture may be continually introduced into the bioreactors 22 during the growth phase 12, such that the water and algae mixture within the bioreactors 22 is continually supplied with the desired concentration of carbon dioxide. The carbon dioxide and air mixture may be intermittently introduced into the bioreactors 22 during the growth phase 12, such that the amount of carbon dioxide within the water and algae mixture is maintained within an acceptable range conducive to the growth of the desired algae.

The light sources 40 of the bioreactors 22 may be configured to continually direct light into the water and algae mixture within the bioreactors 22 or to turn on and off to mimic the day and night cycle of natural sunlight. The light sources 40 may be configured to emit light in a flashing pattern generally conducive to growth and photosynthesis of the algae. If the light sources 40 are light-emitting diodes (LED), the light sources may be configured to emit flashes of light in a pattern of very short, successive flashes. The light sources 40 may be configured to emit 3 to 5 flashes of light per second, preferably 4 flashes of light per second, with each flash of light including light in the wavelength range from 700-800 nanometers. Alternatively, the light sources 40 may be configured to emit from 90 to 110 flashes of light per second, preferably 100 flashes of light per second, with each flash of light having a flash duration of approximately 10 microseconds.

The maintenance of the bioreactors 22 during the growth phase 12 results in an algal density greater than that of the feedstock of algae initially supplied to the bioreactor 22. During the growth phase 12, the algae within the bioreactors 22 may be permitted to amplify to an algal density at or approaching the maximum algal density for which conditions conducive to growth of the algae may be maintained. The algae within the bioreactors 22 may be permitted to amplify to an algal density at or approaching an upper limit where further growth of the algae would likely result in death or degradation of a significant portion of the algae within the bioreactor 22. Alternatively, the algae within each bioreactor 22 may be permitted to amplify to a target or desired algal density.

Upon completion of the growth phase 12, the bioreactors 22 each contain a mixture of water, nutrients, and an amplified quantity of algae. This mixture may then be subjected to the optional nutrient removal 14 to rapidly remove nutrients from the algae. The nutrient removal 14 may accomplished via filtration of the contents of the bioreactors 22 by a filter 48 to separate the algae from the water containing the nutrients. For example, in the system 46 illustrated in FIG. 3, each of the bioreactor couplers (not shown) is connected by a first set of pipes 50 to a first processing reservoir 52 sized to hold a portion or the collective contents of the bioreactors 22. A second set of pipes 54 may then pass the mixture through the filter 48 and the liquid filtrate into a second processing reservoir 56.

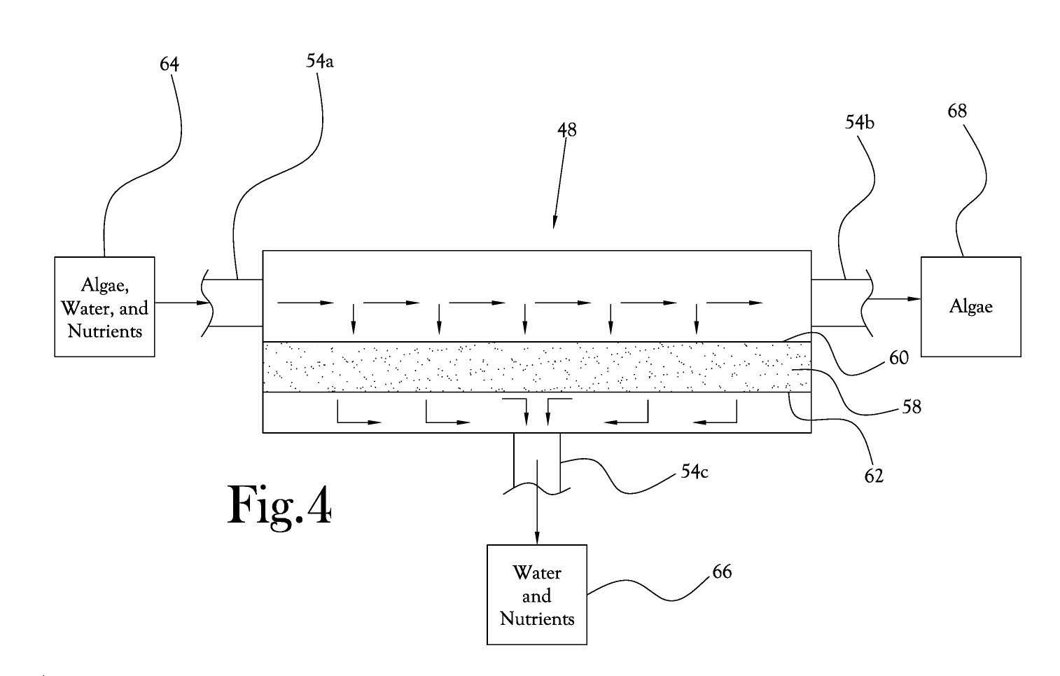

FIG. 4 represents a cross-sectional side view of the filter 48 useful in performing the nutrient removal 14 of the method. The filter 48 may be a “crossflow filter” or “tangential flow filter”, for example. The filter 48 may include a filtration membrane 58 having a retentate side 60 and a permeate side 62, and defining a plurality of pores which are sized to substantially prevent algae cells from passing through the membrane 58, but allow at least a portion of the water containing the nutrients to pass through the membrane 58. The plurality of pores may be less than or equal to ten microns, for example. The filter 48 may be configured such that a mixture of algae, water, and nutrients 64 is directed tangentially across the retentate side 60 of the membrane 58. As the mixture of algae, water, and nutrients 64 travels through the filter 48, positive pressure is maintained on the retentate side 60 relative to the permeate side 62. Thus, a portion of the water containing the nutrients passes through the membrane 58 and forms a permeate 66 of the filter 48. The algae and the portion of water and nutrients which do not pass through the membrane 58 form a retentate 68 of the filter 48.

Referring to FIG. 3 and to FIG. 4, the mixture of algae, water, and nutrients 64 is directed from the first processing reservoir 52, through an input pipe 54a, to a retentate side 60 of the interior of the filter 48. The mixture 64 is then allowed to flow substantially tangential to the retentate side 60 of the membrane 58, whereupon the permeate 66 flows through the membrane 58 as discussed above and is thus separated from the retentate 68. The mixture 64 flowing tangential to the retentate side 60 is maintained at relatively low pressure, such as for example less than one atmosphere of pressure. The retentate 68, including the algae and the portion of water and nutrients which do not pass through the membrane 58, is directed through a first output pipe 54b from the filter 48 to the second processing reservoir 56. The permeate 66, including the portion of the water containing the nutrients which passes through the membrane 58, is directed through a second output pipe 54c to an output of the filter 48. In various embodiments, the permeate 66 is discarded as waste. In other embodiments, the permeate 66 may be retained for use in subsequent iterations of the above-discussed growth phase 12.

Due to the removal by the filter 48 of the portion of the water containing nutrients forming the permeate 66, the retentate 68 of the filter 48 thus contains a higher concentration of algae than the mixture 64 of algae, water, and nutrients fed into the filter 48 from the first processing reservoir 52. Thus, once the retentate 68 is passed through the filter 48 and received into the second processing reservoir 56, additional clean water may be added to the algae via a water source 70. Thus, a mixture may be formed in the second processing reservoir 56 including water, the amplified quantity of algae, and a significantly reduced amount of the above-discussed nutrients.

The amount of water and nutrients removed from the mixture 64 as permeate 66 as a result of passing the mixture 64 through the filter 48 is dependent upon several factors, including, but not limited to, the permeability of the membrane 58, the surface area and length of the flow path across the retentate side 60 of the membrane 58, the pressure differential maintained between the retentate side 60 and permeate side 62 of the membrane 58, and the rate of flow of the mixture 64 through the filter 48, among other factors. The filter 48 may be configured to allow the removal of a significant portion of the water and nutrients from the mixture 64 in a single pass through the filter 48. The nutrient removal 14 may be completed by performing a single pass of the mixture 64 through the filter 48, followed by a single iteration of adding clean water in the second processing reservoir 56 in order to form a mixture of algae and water absent a significant portion of the supplied nutrients.

Alternatively, the nutrient removal 14 may include multiple iterations of alternating filtration and water addition operations to form the mixture of algae and water absent the significant portion of the supplied nutrients. For example, in FIG. 3, the second processing reservoir 56 is connected, via a third set of pipes 72, to the first processing reservoir 52. Thus, once the initial mixture 64 of algae, water, and nutrients is passed through the filter 48 a first time to remove the portion of water containing the nutrients, and once clean water is added to the algae in the second processing reservoir 56, the resultant mixture of water, algae, and the reduced quantity of nutrients may be directed back to the first processing reservoir 52, whereupon the mixture may again be passed through the filter 48 in order to remove additional water and nutrients from the mixture. Additional clean water may then be added to further dilute the nutrients within the mixture following the second pass through the filter 48. This process of iterative filtration and water addition may be repeated until a desired portion of the supplied nutrients is removed from the mixture, thereby completing the nutrient removal 14. The process of iterative filtration and water addition may be repeated until the desired removal of nutrients is accomplished.

Following the nutrient removal 14, the mixture of algae and water is subjected to the stress phase 16, in which the algae is maintained in a relatively low-nutrient, relatively high-salt, or both environment under conditions which are otherwise conducive to photosynthesis and growth of the algae. For example, in FIG. 3, following the nutrient removal 14, the mixture of algae and water is returned to the bioreactors 22 via a fourth set of pipes 74. Similarly to the growth phase 12, the mixture of water and algae is exposed to light via the light sources 40 within the bioreactors 22, and is provided with a supply of carbon dioxide via a mixture of carbon dioxide and air introduced to the bioreactors 22. During the stress phase 16, the algae within the bioreactors 22 may be exposed to light levels in the range of 100-800 micromoles per square meter per second or more, and temperatures from 20 to 36 degrees Celsius (approximately 68 to 96.8 degrees Fahrenheit). Alternatively, during the stress phase 16, the bioreactors 22 may be maintained at a temperature from 25 to 27 degrees Celsius. In this nutrient depleted but photosynthesis conducive environment, the algae within the bioreactors 22 is encouraged to produce astaxanthin-rich cysts within the algae cells.

The growth phase 12, the nutrient removal 14, and the stress phase 16 are configured to result in minimal damage or degradation to the cells of the algae within the mixture. For example, during the nutrient removal 14, the filter 48 may be configured to maintain flow of the algae cells across the membrane 58 and to discourage the algae cells from becoming lodged in the membrane 58, thereby damaging the cells. Furthermore, the nutrient removal 14 allows for rapid removal of nutrients and other contaminants from the mixture of water and algae, thereby minimizing the amount of time the algae is deprived of water and/or nutrients, and limiting the amount of time the algae is exposed to contaminants, prior to the stress phase 16. Thus, following the removal 14, the mixture of algae and water subjected to the stress phase 16 includes a relatively high quantity of healthy algae cells with a minimal amount of dead or dying algae cells or other contaminants. Accordingly, during the stress phase 16, a relatively high yield of astaxanthin is produced by the healthy algae as compared to prior conventional processes.

Regarding FIG. 3, additional devices may be provided in the system 46 in various configurations to facilitate movement of the algae, water, and nutrient mixtures between the bioreactors 22, the first and second processing reservoirs 52, 56, and the filter 48, and to facilitate containment of the algae, water, and nutrient mixtures within the bioreactors 22 and the reservoirs 52, 56. For example, valves (not shown) may be provided proximate leading ends of each of the first, second, third, and fourth sets of pipes 50, 54, 72, 74 and configured to regulate flow through the respective pipes. The various valves may be adjusted between open and closed positions such that flow through each of the pipes 50, 54, 72, 74 may be allowed or disallowed. Additionally, a drive mechanism may be provided to drive flow of the algae, water, and nutrient mixtures through the various pipes 50, 54, 72, 74 when the valves associated with such pipes are in an open position.

In FIG. 3, the bioreactors 22 and the reservoirs 52, 56 define a substantially airtight interior, and a source of pressurized air is provided in fluid communication with the interiors of each of the bioreactors 22 and the reservoirs 52, 56. Thus, pressurized air may be selectively introduced to at least one of the bioreactors 22 or the reservoirs 52, 56 to drive flow of the algae, water, and nutrient mixtures through the pipes 50, 54, 72, 74 associated therewith. For example, an air pump (not shown) may be provided in fluid communication with the interior of the first processing reservoir 52. Each of the bioreactors 22 may then be configured such that, upon opening the valves associated with the first set of pipes 50, the mixture of algae, water, and nutrients drains from the bioreactors 22 into the first processing reservoir 52. Thereafter, the valves associated with the first set of pipes 50 may be closed, and the valves associated with the second set of pipes 54 may be opened, such that flow of the algae, water, and nutrient mixture is allowed through only the second set of pipes 54. Air may then be pumped into the first processing reservoir 52 in order to urge the algae, water, and nutrient mixture through the second set of pipes 54, thus moving the algae, water, and nutrient mixture through the filter 48. Likewise, once the filtered algae and water is received within the second processing reservoir 56 and the additional water added thereto, the valves associated with the second set of pipes 54 may be closed, and the valves associated with the third or the fourth set of pipes 72, 74 may be opened to allow the flow of algae and water back to the first processing reservoir 52 or to the bioreactors 22, respectively. Thereafter, air may be pumped into the second processing reservoir 56 in order to urge the algae and water mixture through either the third or fourth set of pipes 72, 74, thus moving the algae and water mixture to the desired destination.

Other devices suitable for use in directing the algae, water, and nutrients throughout the system 46 may be used. Suitable pumps may be provided to facilitate transfer of the water and algae mixture to the various stations throughout the system 46. For example, a plurality of peristaltic pumps are provided throughout the system 46 to pump the water and algae mixture to the various stations therein.

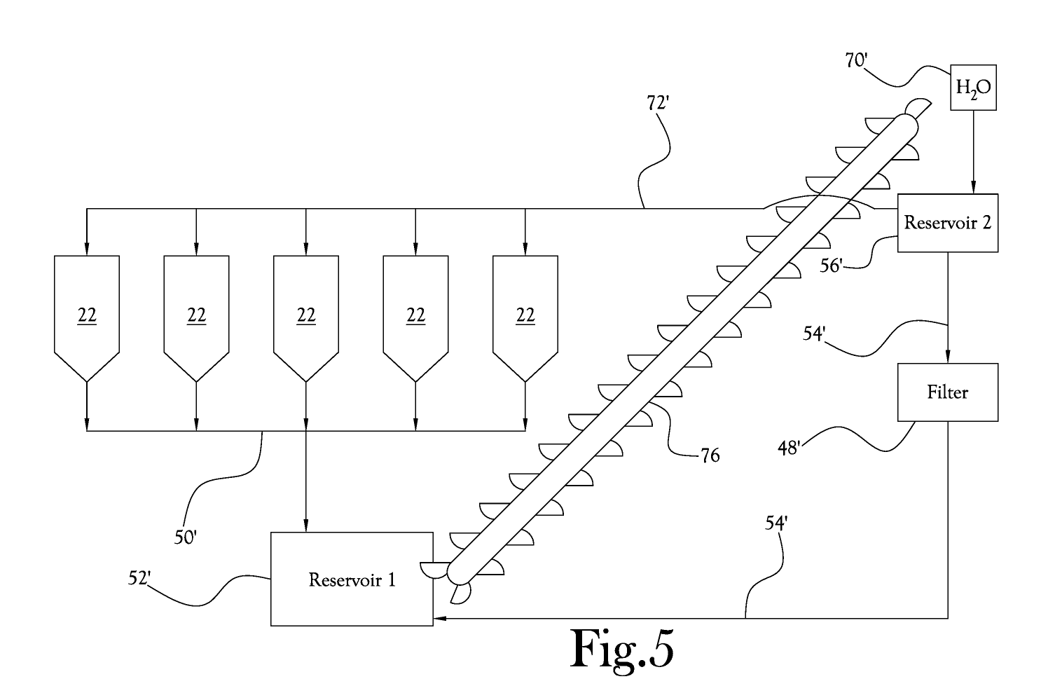

FIG. 5 is another schematic representation of a system that may be used to accomplish several operations of the method. The first processing reservoir 52′ is situated at a lower hydraulic gradient in relation to the bioreactors 22, such that the bioreactors 22 are collectively configured to drain into the first processing reservoir 52′ upon opening the necessary valves to allow the contents of the bioreactors 22 to flow through their respective lower end openings (not shown) and through the first set of pipes 50′. The second processing reservoir 56′ is situated at a higher hydraulic gradient in relation to both the bioreactors 22 and the first processing reservoir 52′.

A conveyor 76, such as for example a bucket conveyor or the like, is provided in communication with the first and second processing reservoirs 52′, 56′, such that, during the nutrient removal phase 14, the conveyor 76 may receive the mixture of water, algae, and nutrients from the first processing reservoir 52′ and transfer the mixture to the second processing reservoir 56′. A second set of pipes 54′ is in communication with a lower end of the second processing reservoir 56′ and is configured, upon opening of suitable valves associated therewith, to allow the contents of the second processing reservoir 56′ to drain therefrom and to direct such contents through the filter 48′ before directing the filtered contents back to the first processing reservoir 52′.

The conveyor 76 may then return the filtered contents to the second processing reservoir 56′ for addition of clean water thereto via the water source 70′. A third set of pipes 72′ is in communication with the lower end of the second processing reservoir 56′ and is configured, upon opening of suitable valves associated therewith, to allow the contents of the second processing reservoir 56′ to drain therefrom and to direct such contents back to the bioreactors 22. Thus, in FIG. 5, transfer of the mixed water, algae, and nutrients to and from each of the various stations in the system 46′ throughout the growth phase 12, the nutrient removal 14, and the stress phase 16 may be accomplished solely via the conveyor 76 and in conjunction with gravitational forces acting upon the mixture.

The harvest phase 18 includes separation of the astaxanthin-rich algae from at least a significant portion of the water in the stress phase 16 mixture. For example, with regard to FIG. 3 and to FIG. 4, upon completion of the stress phase 16, the mixture including water and astaxanthin-rich algae is transferred from the bioreactors 22 to the first processing reservoir 52, where the mixture is passed at least once, preferably multiple times, through the filter 48. Similarly to the nutrient removal 14, upon passing the mixture through the filter 48, a significant portion of the water in the mixture passes through the membrane 58 and exits as permeate through the second output pipe 54c to an output of the filter 48—while the astaxanthin-rich algae travels along the retentate side of the membrane 58 and exits as retentate through the first output pipe 54b. Alternatively, upon completion of the stress phase 16, the mixture including water and astaxanthin-rich algae may be press-filtered to force a significant portion of the water from the algae. Alternatively, the mixture including water and astaxanthin-rich algae is moved to a centrifuge, where the mixture is subject to centripetal acceleration in order to separate the astaxanthin-rich algae from the water.

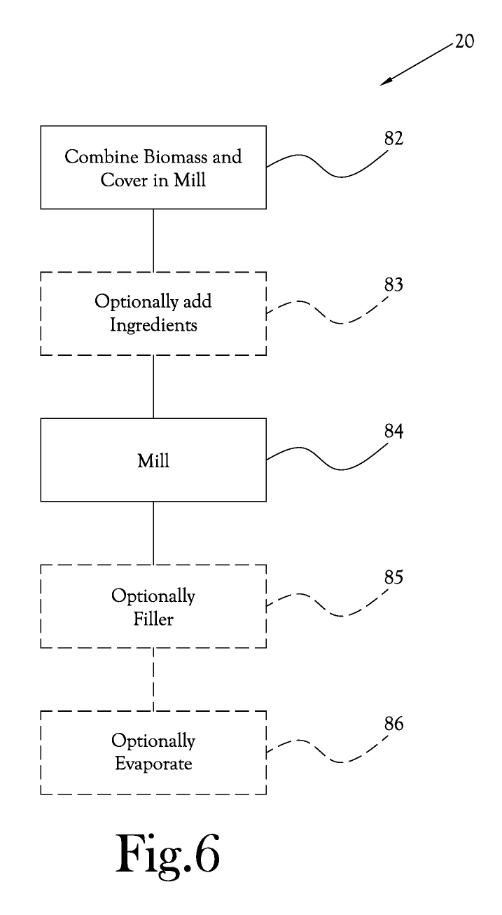

FIG. 6 represents the method 20 of extracting essential oils from an essential oil rich biomass. The essential oil rich biomass introduced to the mill may be habanero peppers including capsaicin compounds. The essential oil rich biomass introduced to the mill may be cannabis plants including cannabinoid compounds. The essential oil rich biomass introduced to the mill may be Haematococcus pluvialis algae including astaxanthin, Phaffia rhodozyma yeast including astaxanthin, or cells rich in omega 3 fatty acids, such as eicosapentaenoic acid (EPA) and/or docosahexaenoic acid (DHA).

Plant-based biomasses from which essential oils may be extracted by the method 20 may include Phaedactylum tricornutum, Spirulina, Chlorella, Nannochloropsis, Monodus subterraneus, Crypthecodinium cohnii, Schizochytrium, Thraustochytrium aggregatum, sunflower seeds, Ulkenia sp., and the like. Plant-based biomasses from which essential oils may be extracted by the method 20 also may include peppers, such as jalapeno, chili, and others, garlic, tomatoes including lycopene, hop concentrates, wheat including germ oil, ginseng including ginsenoside, grape seeds including oils, tobacco, and the like. Animal-based biomasses from which essential oils may be extracted by the method 20 may include green shell mussels, other shell fish, shark cartilage, collagen extracts, DHA and EPA from fish, egg yolks including lecithin, and the like.

In 82, the biomass and a cover are combined in a mill. One form of such mill is an attrition mill with a vessel having a generally annular interior, a shaft extending along a central axis of the vessel, a plurality of paddles extending orthogonally from the shaft, and a plurality of media including balls of ceramic or other substantially hard, non-reactive material. The mill is preferably configured such that the shaft and associated paddles may be rotatably driven about the central axis within the vessel to agitate the media, biomass, and cover. The shaft and associated paddles are preferably configured to be capable of being driven at relatively high revolutions per minute (“RPM”), e.g. 50-1,200 RPM, preferably 50-800 RPM. Preferable milling media include zirconia and alumina materials, which may be sized at approximately 3 to 6 millimeters in diameter. The 3 millimeter milling media provide an approximately 70 micron contact area on impact, for example. The mill is preferably configured with a jacket that can include a liquid to regulate the interior of the mill. The mill is preferably configured to operate at or near atmospheric pressure.

The forces applied by the ball media to the biomass are fundamentally different than those applied by conventional bulk grinding or SCCO2 techniques. Unlike with bulk grinding, crushing, chopping, and the like, ball milling is fundamentally different due to the high shear forces created simultaneously with impact force. These forces also are continually applied to the biomass in the presence of the cover, allowing for solubilization of the essential oil upon release from the physical structure of the biomass. Furthermore, the shear and impact forces applied to the biomass are not substantially decreased as the average particulate diameter of the biomass is reduced as would be the case for other methods where smaller particulates are shielded from continued size reduction by the larger particulates.

Unlike SCCO2 extraction techniques that apply a static pressure to the biomass during the extraction, the shear and impact forces applied to the biomass particulates increase as the average diameter of the biomass particulates decrease. The physical structures of biomass materials also have a high resistance to the substantially static pressure applied during SCCO2 extraction, but have a substantially lower resistance to the shear forces applied by the ball media during the milling. Neither does the SCCO2 method apply mechanical agitation or “stirring” to the biomass, which allows for the essential oil in the interior of the biomass to be shielded from extraction. Thus, while the pressure within the attrition mill is significantly less than the pressure within the SCCO2 extraction vessel, the “pressure” in the form of impact and shear forces the attrition mill applies to the biomass is significantly greater.

The mill preferably includes internal milling and containment surfaces fabricated from materials that are substantially non-reactive to essential oils such that an essential-oil-rich biomass may be contained and milled within the mill with limited, and preferably no, contact with surfaces other than the non-reactive surfaces within the mill. In addition or instead of being fabricated from materials that are substantially non-reactive to the essential oils, the shaft, paddles, and interior surfaces of the mill vessel may be coated with a non-reactive coating, such as for example silicon nitride, polytetrafluoroethylene, or the like. Other types of milling apparatus defining other configurations of milling and containment surfaces may be used.

The cover is selected to limit exposure of the essential oil within the essential-oil-rich biomass to oxygen and other reactants in the atmosphere during the extraction process 20. The cover is preferably a polar solvent. The cover preferably does not include liquid carbon dioxide or other “liquids” that are not liquid at atmospheric pressure. The cover preferably may be readily removed from the extracted essential oil and is edible. For example, the cover may be a volatile alcohol, preferably ethanol, sufficient to substantially coat the essential-oil-rich biomass. The cover may be other volatile solvents, such as acetone or toluene, but these are more difficult to remove from the extracted essential oil and are not edible. Instead, or in addition to alcohol, the cover may be a hydrophobic, lipid-based oil derived from animal or vegetable sources. Thus, the cover may be selected from the group consisting of olive oil, sunflower oil, fish oil, vegetable oil, ethanol, and combinations thereof. The cover may be accompanied with an oxidatively inert gas, such as for example nitrogen, argon, and the like. Thus, depending on the essential oil, a polar alcohol, a lipid-based oil, an accompanying oxidatively inert gas, or a combination thereof may be used as the cover.

The biomass may be introduced to the mill followed by the cover, the cover may be introduced to the mill followed by the biomass, or the biomass and cover may be added substantially simultaneously. For example for astaxanthin, with reference to FIG. 3, following the stress phase 16, and after the water is removed from the mixture including water and astaxanthin-rich biomass by the filter 48 (thus, the harvesting 18 of FIG. 1), the dried astaxanthin-rich algae is directed to the second processing reservoir 56. The cover is then added to the dried astaxanthin-rich algae within the second processing reservoir 56, whereupon the combination is introduced into the mill.

In 83, additional ingredients optionally may be added to the mill for milling and/or mixing with the biomass and the cover. For example, when an astaxanthin-rich biomass is provided, the additional ingredient may include at least one omega 3 fatty acid, such as eicosapentaenoic acid (EPA) and/or docosahexaenoic acid (DHA). Thus, at least one additional biomass including algae and/or bacteria of the type rich in eicosapentaenoic acid (EPA) and/or docosahexaenoic acid (DHA) may be added to the mill. Other additional ingredients may be added to the mill.

Following the combination of the biomass with the cover 82, and the optional addition of one or more additional ingredients 83, the mill is activated 84. During the milling 84, the contents of the mill are reduced in size as the physical structures and cells of the biomass are broken open to release the essential oils. The milling 84 may also reduce the average particulate diameter of the released essential oils under cover of the cover. Thus, if an insoluble aggregate of the essential oil is released from the biomass or forms during milling, the milling 84 can reduce the size of the aggregate to a molecular level where the molecules constituting the essential oil are soluble in the cover. Furthermore, the cover discourages oxidation or other atmospheric-based contamination of the essential oils during the milling 84.

When astaxanthin-rich algae is milled, the milling 84 encourages diminution of the released cysts and aggregated astaxanthin molecules into particulates wherein the average particulate diameter is reduced to less than 3 microns, to less than 35 nanometers, or to less than 30 nanometers. The aggregated or dimerized astaxanthin particulates may be milled to have a size approximately equal to a single astaxanthin molecule, thus to a non-aggregated, monomeric state.

During the milling 84, the contents of the attrition mill may be maintained at a cooler than room temperature. Lower temperatures may further discourage oxidation of the released essential oils. Such cooling may be provided by water-cooling the mill during the milling 84. However, such below room temperature during the milling 84 is not required, and in fact, most biomasses benefit from milling at higher than room temperature to increase the rate of extraction. For example, during the extraction of cannabinoids from cannabis biomass, the mill may be maintained at an internal temperature from 50 to 90 degrees, preferably from 60 to 80 degrees, and more preferably from 67 to 73 degrees Celsius. Preferably, the internal temperature of the mill is controlled to be close to the boiling point of the room temperature liquid or liquids forming the cover.

In the milling 84, a mixture in the form of a slurry is produced including the milled biomass byproduct, essential oils, any optionally added additional ingredients, and the cover. When the cover is or includes a solvent for the essential oil, the milling 84 of the biomass results in a solution, with the essential oil as a solute and the cover or a portion of the cover as the solvent. Thus, the cover may be a solvent for the essential oil or a multi-phasic mixture including a solvent for the essential oil. Together, the cover and the milled biomass byproduct in combination form what could be considered a slurry during milling.

For example, milling of the astaxanthin-rich Haematococcus pluvialis algae results in shearing of the astaxanthin into very small particulates that dissolve as the solute to form a solution with the ethanol cover or ethanol portion of the cover as the solvent. Suspended or mixed with the ethanol solution are the undissolved portions of the milled biomass byproduct.

When the milling 84 is complete, the mixture may be removed from the mill and utilized by an end user, or packaged for subsequent use by an end user. Alternatively, filtration 85 may be used to collect the solution from the milled biomass byproduct. Porosity, centrifugation, settling, filter pressing, or other “filtration” method may be used to collect the solution. If the solution is removed, the remaining milled biomass byproduct may be washed with additional aliquots of solvent to remove additional solute that failed to dissolve in the cover during the milling. As a relatively small amount of cover may be used in relation to the biomass, essential oils may be released from the biomass that cannot be solvated due to saturation of the cover at the selected temperature. Similarly, additional solvent may be added to the mixture before the filtration 85 to dissolve additional solute. The solvent may be the milling cover or a different solvent for the desired solute.

The cover may be a material which is generally edible by fish, livestock, or other animals. In this instance, upon completion of the milling 84, the mixture may be removed and packaged for further use in, for example, marine or agriculture feed products or the like. In this instance, the cover may be an oil or an oil combined with a solvent. If the solvent is not desired in the feed, the solvent may be evaporatively removed from the oil, thus leaving the essential oil solute mixed with the oil cover. Heat, vacuum, or the like may be used to evaporatively remove the solvent from the oil and solute mixture.

Alternatively, the mixture or solution may be optionally evaporated 86 to remove substantially all of the cover. For example, when the cover is liquid ethanol, upon completion of the milling 84, the mixture may be transferred to a vacuum dryer, whereupon the ethanol is evaporatively removed from the mixture to form a solid, granular product including the essential oil particulates and byproducts of the milled biomass. In another example, when the solution is removed from the mixture by the filtration 85, the solvent of the solution may be evaporated from the solute and the solute dosed into oils at desired concentrations in a pure state or dried and used as very pure, high concentration essential oil.

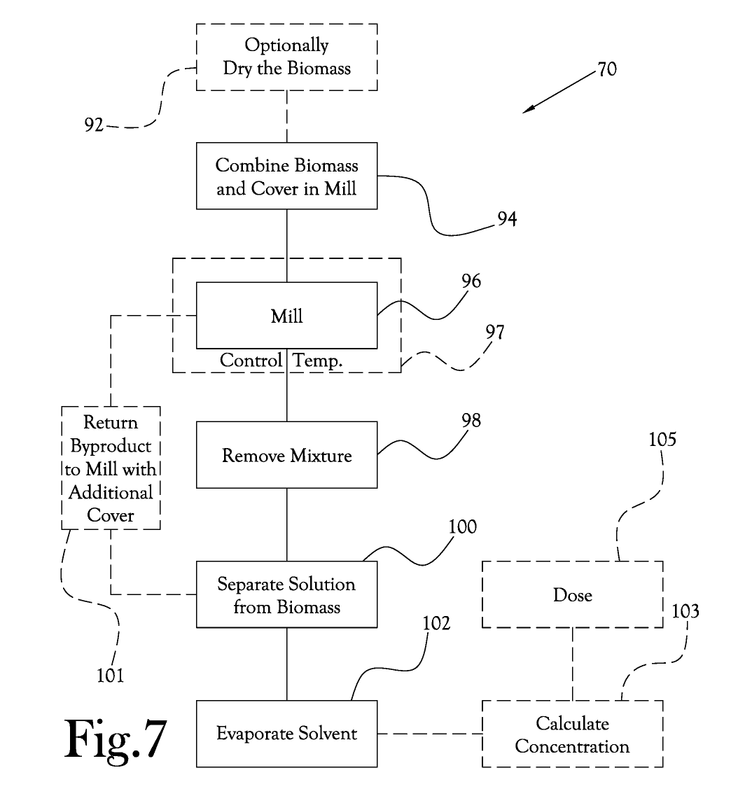

FIG. 7 represents a method 70 of extracting essential oils from a biomass. The essential oil may be astaxanthin from a biomass of Haematococcus pluvialis algae, capsaicin compounds from Habanero or other peppers, cannabinoids from cannabis, or another essential oil from a biomass. Prior to combining the biomass and the cover in a mill 94, the biomass may be dried. Drying 92 may include placing the water including biomass in a vacuum dryer, where the water including biomass is subjected to increased temperature and low pressure to remove substantially all or a portion of the water from the biomass. Pressures from −100 to −800 Torr may be used during the drying 92, with pressures in the −700 Torr range being preferred. The low pressure may be used with or without the increasing the temperature above room temperature, and for some biomasses, the low pressure may be used with temperatures lower than room temperature. The drying 92 may be performed other than through vacuum drying, with the intent being to remove a desired portion or preferably substantially all of the water from the biomass, while reducing oxidative or other degradation of the desired essential oil/s.

Once the optional drying 92 is completed, the biomass is combined in an attrition mill with a cover 94. The mill may be an attrition mill, such as the type manufactured by Union Process® and marketed using the model number “5-1.” The attrition mill may have a volume of approximately 7 liters, be lined with TEFZEL®, have silicon nitride or TEFZEL®-coated paddles, and contain approximately 8 kilograms of 3-millimeter media balls fabricated from silicon nitride, zirconia, or other material compatible with the collection of the desired essential oil. For astaxanthin, for example, approximately 800 milliliters of ethanol may be combined in the attrition mill with approximately 600 milligrams of dried Haematococcus pluvialis algae biomass. For other biomasses, the ratio of cover to biomass may differ.

After the combination 94, the mill is operated 96, such that the biomass within the mill, and the essential oil fraction contained therein, is milled. The paddles in the attrition mill may be rotated at approximately 400 revolutions per minute (RPM) for approximately 20 minutes, for example. Different rotational speeds and milling durations may be selected in view of the biomass, operation temperature, and cover. Throughout the milling 96, the temperature of the contents of the mill optionally may be controlled 97. As previously discussed, the attrition mill may be equipped with a water jacket or other heat exchange system configured to provide temperature control to the contents of the mill.

As previously discussed, production of astaxanthin within Haematococcus pluvialis algae cells occurs through the growth of relatively hard, dense, astaxanthin-rich cysts within the algae cells. The milling 96 results in the cysts within the mill being subjected to relatively high-energy shear forces by the motion of the media balls within the mill. Thus, throughout the milling 96, very small particulates of the carotenoid fraction are sheared from the algal cysts. Such very small particulates of astaxanthin may enter solution as a solute within the cover solvent. Thus, the milling 96 results in a mixture within the mill of crushed or sheared solids of Haematococcus pluvialis algae byproduct, together with a solution including a solvent and a solute. The solute is mostly carotenoid and includes from 60% to 98% astaxanthin and canthaxanthin by weight, preferably from 75% to 98% astaxanthin and canthaxanthin by weight, and more preferably from 82% to 88% astaxanthin and from 3% to 7% canthaxanthin by weight. The resulting carotenoid solute may be considered an oleoresin and is lipophilic.

Unlike for astaxanthin, algal cysts are not present in plants. However, the cell walls of seeds, stalks, bark, and other cellular structures may be similarly difficult for conventional, especially SCCO2, methods to extract. Regardless of the type of cellular structure involved, the milling 96 will free the essential oils from the biomass and allow solvation in the solvent.

Following the milling 96, the mixture of milled biomass byproduct and solution may be removed 98 from the mill mechanically and/or by rinsing the various components of the mill with additional solvent. Portions of any undissolved essential oil may dissolve in the additional solvent added during the removal 98. Following the removal 98 of the milled biomass byproduct and solution from the mill, the biomass byproduct is separated from the solution in solid-liquid separation 100.

In the separation 100, the milled biomass byproduct solids are substantially separated from the liquid solution. The solids may be removed by settling, skimming, decanting, or otherwise removing the milled biomass byproduct solids from the solution. Preferably, greater than 90% and more preferably, approximately 99% of the milled biomass byproduct solids are removed from the solution. For Haematococcus pluvialis algae, for example, the mixture of the milled algae biomass byproduct solids and the solution may be subjected to forced settling, for example, by running the mixture in a continuous centrifuge. A centrifuge also may be used for peppers and cannabis.

After the separation 100, the remaining milled biomass byproduct solids may contain additional undissolved essential oil. This remaining milled byproduct solids including undissolved essential oil may be returned in 101 to the attrition mill with additional cover for additional milling. The milling 96, optional temperature control 97, removal 98, and separation 100 may be repeated, with or without the addition of un-milled biomass, to produce additional solution. This “remilling” of the previously milled biomass byproduct solids and undissolved essential oil may be repeated until a desired amount, or substantially all, of the essential oil present in the biomass is dissolved in solution.

In solvent evaporation 102, the solvent content of the solution is reduced. The solvent evaporation 102 results in an essential oil solute product which is either highly concentrated in the remaining solvent or is essentially pure solute. The solvent evaporation 102 may be implemented with a vacuum dryer, distillation, vacuum distillation, and the like to remove a majority of the ethanol from the solution. Any solvent removal technique may be used that does not substantially degrade the essential oil through oxidation or other pathways. When the solvent is ethanol, distillation may be used to remove the volatile alcohol solvent. When the solvent is an oil or other less-volatile liquid, solvent content reduction may be facilitated by heat.

The essential oil concentrate resulting from the solvent reduction 102 is a concentrated solvent and essential oil solution, suspension, or solid/liquid mixture. The solvent evaporation 102 may be continued until the essential oil concentrate becomes a waxy paste. The solvent evaporation 102 may be continued to remove substantially all of the solvent to provide a solid. The solid may be mixed in a high energy mixer to render the essential oil into a powder. In either case, the resultant essential oil concentrate can be analyzed and weighed and the weight used to determine or estimate the concentration 103 of essential oil in the concentrate. Thereafter, the essential oil concentrate can be dosed 105 into edible oils, for example safflower oil or cod liver oil, to achieve a desired concentration of essential oil in the edible oil.

The method 70 may remove up to 98% by weight of the essential oil from the originating biomass. Essential oil recovery preferably is at least 50%, preferably at least 70%, and more preferably at least 85% by weight in relation to the essential oil weight in the originating biomass.

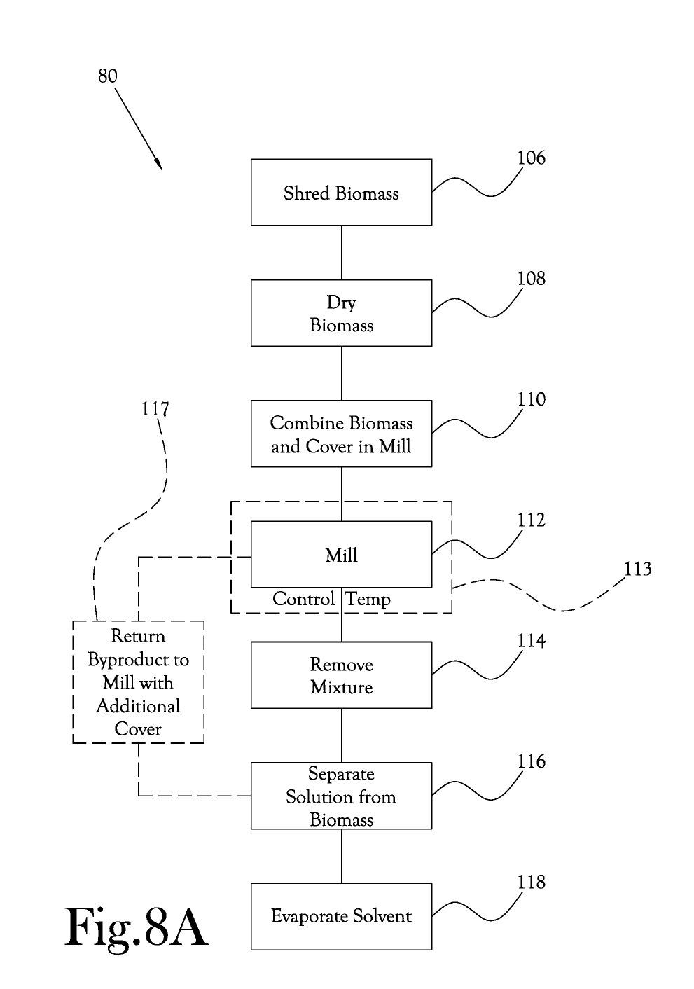

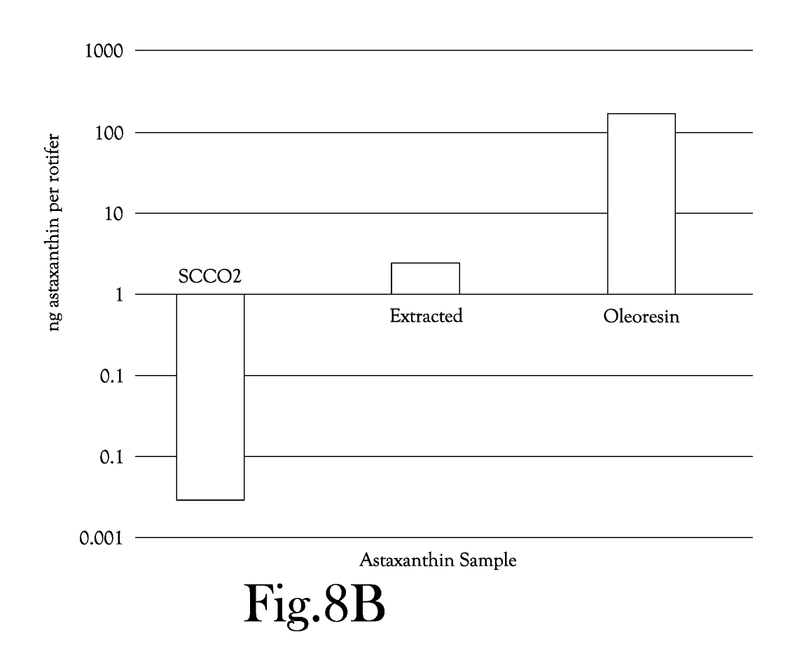

FIG. 8A represents a method 80 of extracting essential oils from a biomass. In 106, a biomass rich in capsaicin compounds (capsaicin and/or dihydrocapsaicin) or cannabinoids is shredded. The biomass may include habanero peppers, black peppers, paprika peppers, jalapeno peppers, chili peppers, other peppers, combinations of peppers, or cannabis plants or cannabis plant parts. In 108, the biomass is dried. Drying may be performed in a vacuum oven with heat or near room temperature, under approximately −740 torr of vacuum. Other drying techniques that do not significantly oxidatively or otherwise degrade the desired essential oil may be used. While the figure visually represents the shredding 106 being performed before the drying 108, the drying 108 may be performed before the shredding 106, depending on the biomass and water content of the biomass.

In 110, the shredded and dried biomass is introduced to a mill with a cover in which the desired essential oil is at least partially soluble. As previously discussed, the cover may be a solvent for the essential oil or a combination of solvent with additional liquids or gases that assist in reducing oxidation of the essential oil during milling. The mill is operated in milling 112 to break the cells of the plants, seeds, fruits, and other structures and release the essential oils into the solvent. As the seeds of habanero peppers, for example, may contain twice as much capsaicin compounds as the shell of the pepper, the milling 112 can break the hard pepper seeds similarly to breaking the Haematococcus pluvialis algae cysts. Similarly, the tough cell walls of cannabis or tobacco stalks may be broken. The mill is operated until the average particulate diameter is less than that obtained by conventional grinding or shredding. In one instance, the mill may be operated until the average particulate diameter is in the 8 micron range to make the biomass suitable for consumption by aquatic filter feeding organisms having a “mouth” in this size regime. In other instances, milling may be continued until the average particulate diameter of the essential oil is from 100 nanometers to 100 microns, from 100 nanometers to 30 microns, or from 100 nanometers to 10 microns. Throughout the milling 110, the temperature of the contents of the mill optionally may be controlled 113. As previously discussed, the attrition mill may be equipped with a water jacket or other heat exchange system configured to provide temperature control to the contents of the mill. Higher than room temperature milling is often preferred. However, depending on the energy generated within the mill during milling, cooling may be required to maintain the temperature in the mill below the boiling point of the cover.

In removal 114, the milled biomass byproduct, in this instance, milled peppers or plant parts, and solution including the dissolved capsaicin, cannabinoid, or tobacco compounds is removed from the mill. Additional solvent may be used during the removal 114 to dissolve additional essential oil into the solvent and assist in flushing the milled biomass byproduct solids from the mill. As previously discussed, the solvent may be an alcohol, preferably ethanol, an oil, or a combination thereof. In separation 116, the milled biomass byproduct solids are separated from the solution as previously discussed. If desired, the milled biomass byproduct solids may be re-milled in 117. In solvent evaporation 118, a portion or substantially all of the solvent is evaporated. The resulting essential oil compound concentrate may be used as-is, or dosed into edible oils, for example safflower oil, to achieve a desired concentration of capsaicin and/or dihydrocapsaicin, or CBD and/or THC in the oil. The resulting essential oil compound also may be redissolved in solvent and the constituent essential oils separated using column chromatography or similar technique. For example, in the case of cannabinoids, the CBD may be separated from the THC through column chromatography.

FIG. 8B is a graph showing the bioavailability of astaxanthin prepared using a conventional SCCO2 technique in comparison to the described methods, with the “extracted” being the bioavailability of the astaxanthin remaining in the biomass after milling and multiple ethanol extractions, and the “oleoresin” being the astaxanthin product produced after evaporation of the ethanol solvent from the essential oil solute. Of note is that the biomass “by-product” has greater astaxanthin bioavailability than the “astaxanthin isolated product” from the conventional SCCO2 technique. As represented in FIG. 8B, the methods of FIG. 7 and FIG. 8A provide a substantial enhancement in bioavailability of the isolated essential oil or oils as the physical structures and cells of the biomass are broken and reduced to a size regime where the essential oils are brought into a solvent to form solution, not extracted as a suspended solid. As the essential oils exist in the solvent as a dissolved solute, bioavailability is not significantly hampered by encapsulation or entrapment with solids or with insolubilized particulates formed when the polar portions of the essential oil molecules bond with each other. Neither are the essential oils substantially oxidized or otherwise degraded during isolation, as is common with conventional methods.

The improved bioavailability of essential oils isolated as described makes the isolated essential oils attractive in various nutraceutical applications, such as for example the preparation of gelatin-based, chewable vitamins—“gummy vitamins.” Adding a powerful antioxidant, such as the above-discussed astaxanthin, to the gummy vitamin is an attractive way to improve the value of the vitamin. However, to do so it is important to be efficient with the overall volume of the gummy vitamin. To date, the inability to concentrate astaxanthin at very high levels has precluded its inclusion in such gelatin-based platforms, which require high concentration preparations of the desired ingredients due to the required dilution with the gelatin.

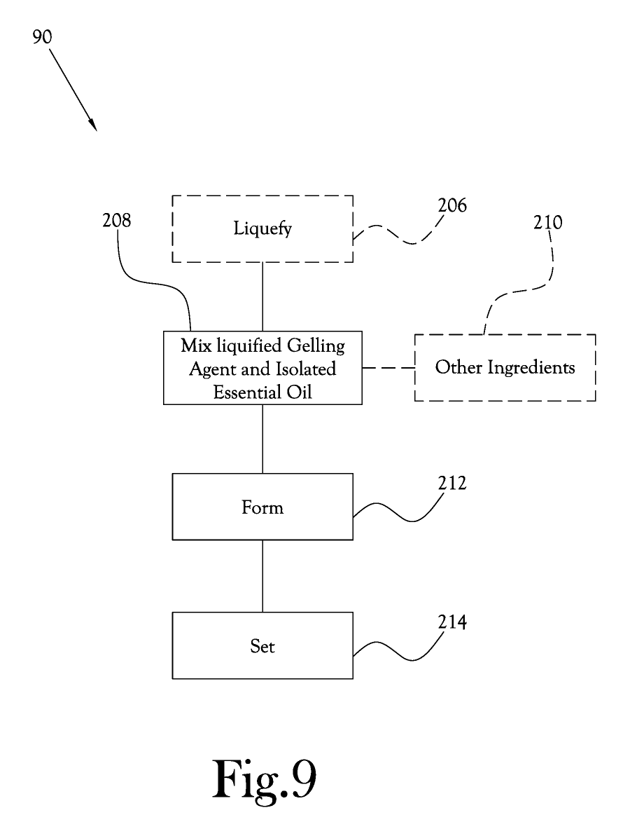

FIG. 9 represents a method 90 of manufacturing a gelatin-based vitamin supplement, thus a chewable gummy vitamin. In mixing 208, the desired quantity of isolated essential oil and optionally liquefied gelling agent, such as collagen based gelatin or pectin, are mixed. The isolated essential oil may be astaxanthin, as previously discussed. The gelling agent may be mixed with a sufficient quantity of hot water or other liquid prior to mixing with the essential oil in optional liquefaction 206. Alternatively, the gelling agent may be mixed with the essential oil, added to the water, and then heated, such that the gelatin dissolves in the water to a sufficient concentration that, once cooled, the gelatin and hot water solution sets to a gel. The specific quantities of gelatin and water may be varied to achieve a set gel of a desired consistency, and the specific ratio of gelatin to water may vary depending upon a number of factors, including but not limited to the water temperature and the amount and consistency of any optional additional ingredients.

In addition to, or in the alternative to, gelatin and pectin, other gelling agents may be used with the understanding that such alternate gelling agents may require alternate procedures for liquefaction, depending upon the specific properties of the gelling agent. For example, in various embodiments, natural gums, starches, agar-agar, or the like, may be used as the gelling agent. While water is the expected liquid for the gelling agent, other liquids may be used depending on the gelling agent.

Optionally, additional ingredients, such as flavoring or coloring agents, preservatives, and/or additional nutraceutical or vitamin ingredients 210, may be added to the gelling agent either before, during, or after the mixing operation 208. In this manner, a liquid precursor to a gummy vitamin is formed including the essential oil, the liquefied gelling agent, and any additional provided ingredients.

The liquid precursor is formed 212 into one or more portions and/or one or more desired shapes. For example, a mold may be used to define a plurality of cavities, each cavity defining a negative of a desired size and shape of a finished gummy vitamin. Portions of the liquid precursor may be poured into each mold cavity, thus forming 212 the liquid precursor into portion sizes and shapes resembling the sizes and shapes of each of the mold cavities. Thereafter, the liquid precursor is allowed to set in 214, thereby forming a finished gummy vitamin.

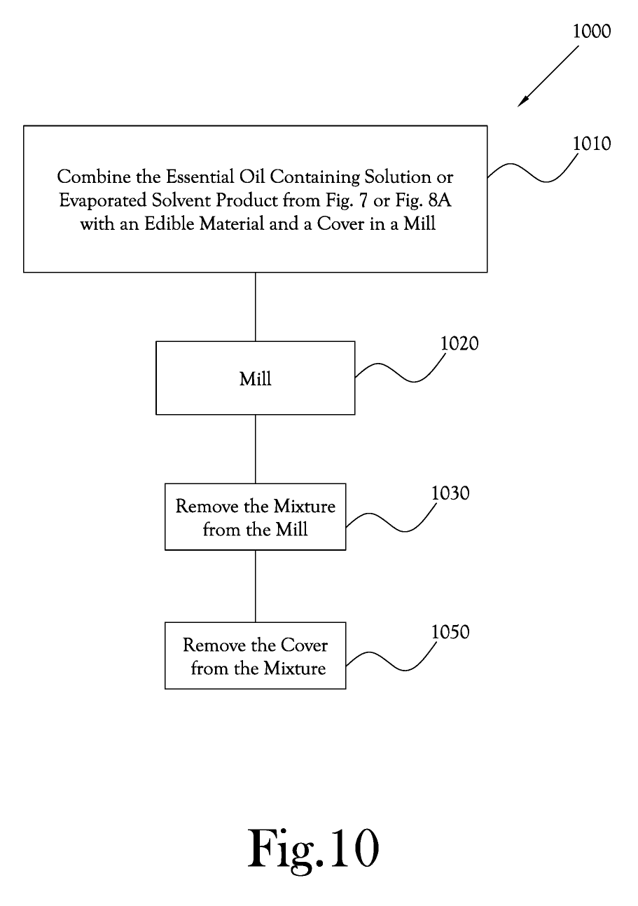

FIG. 10 represents a method 1000 of manufacturing a bioavailable essential oil enriched feed or food additive with a previously extracted essential oil. In combination 1010, the essential oil containing solution or evaporated solvent concentrate from FIG. 7 or FIG. 8A is combined with an edible material and a cover in a mill. The edible material may be a conventional animal feed, or other edible material consumed for an actual or perceived health benefit, such as spirulina algae. Other edible materials, including potato starch, beef heart, and the like may be used. The edible materials are solid or semi-solid materials.

When astaxanthin is the essential oil, such as obtained through the illustrative process of FIG. 7, it is preferable that the edible material has a non-polar character to associate with the non-polar “middle” of the astaxanthin molecule. For astaxanthin, it is more preferable that the edible material has sufficient non-polar character to have a greater affinity for the astaxanthin than the solvent used to extract the astaxanthin from the algal biomass.

In milling 1020, the mill is operated to mix the edible oil with the edible material and to reduce the average particulate diameter of the edible material. Continued reduction in the average particulate diameter of the essential oil particulates also may occur, but this is not the primary objective of the milling 1020. The milling 1020 is continued under conditions and time to optimally transfer the essential oil from the cover to the reduced particulate diameter edible material. In removal 1030, the mixture of cover and essential oil adhered edible material particulates are removed from the mill. This removal optionally may be facilitated with a liquid that does not substantially transfer the adhered essential oil from the reduced particulate diameter edible material. In cover removal 1050, the cover is removed from the reduced particulate diameter edible material. This removal may be performed as previously discussed with regard to settling, filtration, solvent evaporation, or by other techniques, as in this case the product of interest is the solid or semi-solid edible material, not a solute in the cover. The edible material including the essential oil than may be further dried and packaged for sale (not shown).

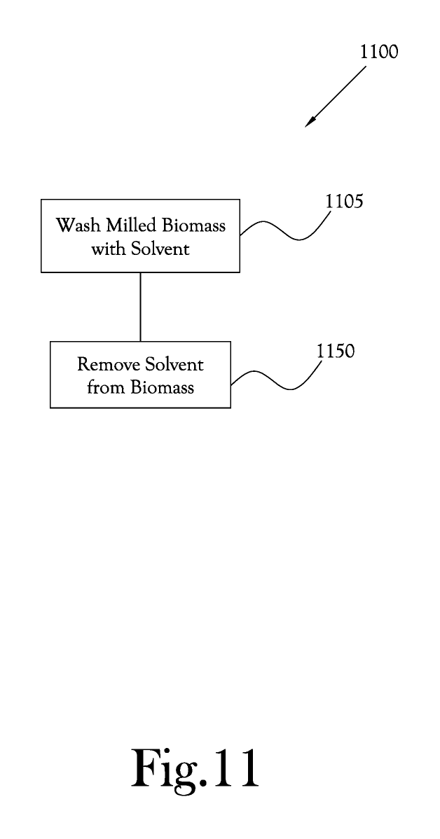

FIG. 11 represents a method 1100 of manufacturing a bioavailable essential oil enriched feed or food additive including the biomass originating the essential oil. As previously discussed, while the essential oil of interest may be present in the originating biomass, the bioavailability of the essential oil if the originating biomass were directly consumed may be exceedingly low. Such exceedingly low bioavailability may be due to the essential oil being concentrated in indigestible physical structures, such as cysts or seeds, or due to the bioactive form of the essential oil as present in the originating biomass being substantially unavailable if released into an aqueous environment, such as in the case of intramolecular bonding between the polar ends of astaxanthin.

In solvent wash 1105, the biomass remaining after solvent removal, such as generally described in separation 100 of FIG. 7 or in separation 116 of FIG. 8A, is washed with additional solvent. The remaining biomass may be washed from 1 to 10 times, preferably from 3 to 7 times, and more preferably from 4 to 6 times. The biomass optionally may be milled, stirred, agitated, heated, and the like with the solvent wash or washes (not shown) to enhance transfer of the essential oil to the solvent.

As the average particulate diameter of the biomass was substantially reduced by the prior milling, the intent is to remove the majority of the essential oil that was released from cysts, seeds, and other physical structures of the originating biomass that is not associated with the reduced particulate diameter originating biomass. Preferably, substantially all of the essential oil that was released from the physical structures of the originating biomass, but that is not associated with the reduced particulate diameter originating biomass is removed with the solvent wash.

When astaxanthin is the essential oil, the non-polar portion of the astaxanthin molecule is believed to allow a fraction of the released astaxanthin to associate with the non-polar, milled particulates of the algae—thus, providing monomeric astaxanthin molecules adhered to the milled algae particulates. The washing of the milled biomass with ethanol allows the astaxanthin not adhered to the milled algae particulates to be recovered for future use, while leaving astaxanthin enriched edible particulates of the originating biomass.

In solvent removal 1150, the wash solvent is removed from the reduced particulate diameter originating biomass. This removal may be performed as previously discussed with regard to solvent evaporation or by other techniques that preserve the bioavailability of the essential oil in the solvent and in the reduced particulate diameter originating biomass.

The following examples illustrate one or more preferred embodiments of the invention. Numerous variations may be made to the following examples that lie within the scope of the invention.

### Example 1: Extracting and Isolating Astaxanthin from a Haematococcus pluvialis Biomass

Haematococcus pluvialis biomass was milled in an attrition mill with food grade ethanol using 3 mm ceramic media (zirconia in this instance) at a 400 RPM paddle speed for approximately 20 minutes at a temperature from about 60 to 70 degrees Celsius. The attrition mill was a Union Process 1S mill and about 500 grams of biomass and 800 mL of ethanol were combined in the mill. After milling for approximately 20 minutes, the ethanol was removed from the mill, new ethanol was added to the mill, and milling was repeated for approximately 20 minutes. The ethanol was again removed, new ethanol added to the mill, and milling repeated. The biomass was removed from the mill and separated from the ethanol solvent by centrifuge. The ethanol solvent was then evaporated from the astaxanthin solute with a vacuum oven using a cold trap to recover the ethanol for reuse. The astaxanthin solutes were recovered as a thick mass of oil resin, or “oleoresin”.

### Example 2: Manufacturing a Bioavailable Astaxanthin Enriched Feed or Food Additive Including the Haematococcus pluvialis Biomass

Haematococcus pluvialis biomass was milled in an attrition mill with food grade ethanol using 3 mm ceramic media at a 400 RPM paddle speed for approximately 20 minutes at a temperature from about 60 to 70 degrees Celsius. The attrition mill was a Union Process 1S mill and about 500 grams of biomass and 800 mL of ethanol were combined in the mill. After milling for approximately 20 minutes, the biomass was separated from the ethanol solvent by centrifuge. The ethanol solvent was evaporated from the astaxanthin solute with a vacuum oven using a cold trap to recover the ethanol for reuse. The recovered ethanol was then recombined with the separated biomass and the mixture centrifuged. This wash and centrifuge process was repeated from 1 to 5 times after the initial separation. While most of the astaxanthin was removed, the milled algae was still stained red in color. The biomass was dried in a vacuum oven to provide an animal feed or supplement.

### Example 3: Enhancing Rotifer Reproduction Rate, Population Growth, and Resistance to Oxidative Stress with Bioavailable Astaxanthin

Three astaxanthin products were tested for their effect on the Brachionus manjavacas rotifer. The first product was unextracted astaxanthin produced by milling H. pluvialis in ethanol and removing the ethanol from the milled material. This first product contained about 3% astaxanthin by weight. The second product was extracted and concentrated astaxanthin obtained generally as described in Example 1. The third product was extracted and dried milled Haematococcus pluvialis obtained generally as described in Example 2. This production contained about 1.1% astaxanthin by weight.

Rotifer reproductive rate was assessed by determining the number of offspring an individual rotifer produced daily during a 72 hour period. Rotifer population density was determined by determining the number of rotifers present in 1 mL of water every 24 hours. Rotifer resistance to oxidative stress was determined by exposing the Rotifers to Juglone and counting the number of surviving rotifers after 24, 48, and 72 hours.

Regarding reproduction rate, the greatest increase for the first product was observed at 80 ug/mL of the first product in water, with an approximate 41% increase in growth rate. The greatest increase for the second product was 32% at 23 ug/mL of the second product in water—with the second product being pre-dissolved in DMSO. The greatest increase for the third product was 43% when 400 ug/mL of the dried biomass was used. Regarding population density, the second product at a 2.3 ug/mL water concentration provided greatest population density and maintained the density longest of the three products. However, at 92 ug/mL the second product proved toxic. Regarding oxidative stress, the first product at 80 ug/mL in water provided an approximately 36% increase in rotifer survival after 72 hours. The products did not significantly increase the lifespan of the rotifers. However, the second product did provide an increase in rotifer swimming speed of approximately 47%, while the first product produced a slight increase.

These results established that enhancing the diets of rotifers with astaxanthin produced by the described methods provides marked increases in reproductive rates, growth, and density, but no appreciable increase in lifespan. The third product provided the greatest increase in rotifer reproduction at the lowest astaxanthin concentration. This is believed attributable to this product having the highest percentage of bioavailable astaxanthin in water, as the astaxanthin is bound to the edible biomass, and as previously discussed, is unlikely to form aggregates in water. Thus, using an edible, non-polar biomass as a carrier for the extracted astaxanthin is believed to maximize the bioavailability of the astaxanthin. In this way, a single portion of the astaxanthin milled from the Haematococcus pluvialis can be used make much greater quantities of astaxanthin enhanced, edible product than the original biomass.

### Prophetic Example 1: Manufacturing an Astaxanthin Enriched Spirulina Supplement

The extracted or extracted and concentrated astaxanthin from Example 1 is combined with dried spirulina algae in an attrition mill. In the case of the concentrated astaxanthin, additional cover is added to the mill. The dry weight of the astaxanthin and dried spirulina combined in the mill approximates the ratio of the astaxanthin to biomass in the bioavailable astaxanthin enriched feed or food additive including the Haematococcus pluvialis biomass produced in Example 2.

The mill is operated with 3 mm ceramic media at a 400 RPM paddle speed for approximately 20 minutes at a temperature from about 60 to 70 degrees Celsius. The attrition mill is a Union Process 1S and about 500 grams of dried spirulina algae and 800 mL of ethanol are present in the mill. The spirulina algae enriched with the astaxanthin is separated from the ethanol solvent by centrifuge and/or by a vacuum oven using a cold trap to recover the ethanol for reuse. The dried algae is recovered to provide a nutritional supplement.

### Prophetic Example 2: Extracting and Isolating Capsaicin Compounds from a Habanero Pepper Biomass

Fruit of the habanero pepper plant is dried and shredded. The shredded pepper fruit biomass is added to the mill with an ethanol cover. During milling, the temperature within the mill is increased to approximately 60 to 70 degrees Celsius. In this instance, of total mill volume, approximately ⅓^{rd} is occupied by the milling media, approximately ⅓^{rd} is occupied by the cover, and approximately ⅓^{rd} is occupied by the biomass. The milling media may be metal or ceramic.

During milling, the mill is closed to the atmosphere, thus allowing slight pressurization from volatilization of the ethanol. However, as the temperature within the mill is maintained at approximately 60 to 70 degrees Celsius and the boiling point of the ethanol cover is 77 degrees Celsius, the internal mill pressure does not exceed 200 kPa. The mill is operated at approximately 400 RPM for approximately 20 minutes. The cover may be removed from the mill, and new cover added for a repeat of the milling cycle. While not required, repeated mill cycles with new cover will increase capsaicin compound recovery.

The mixture of biomass and cover along with any desired cover is then filtered using a centrifuge to remove the remaining biomass from the cover. The cover is then removed from the capsaicin compounds essential oils using a vacuum oven. The cover is recovered in a cold trap for reuse. The resulting essential oils may be used as previously described.

### Prophetic Example 3: Extracting and Isolating Cannabinoid Compounds from a Cannabis Biomass

The leaves, stalks, seeds, flowers, and any combination thereof of the cannabis sativa plant are dried and shredded. The shredded cannabis biomass is added to the mill with an ethanol cover. During milling, the temperature within the mill is increased to approximately 60 to 70 degrees Celsius. In this instance, of total mill volume, approximately ⅓^{rd} is occupied by the milling media, approximately ⅓^{rd} is occupied by the cover, and approximately ⅓^{rd} is occupied by the biomass. The milling media may be metal or ceramic.

During milling, the mill is closed to the atmosphere, thus allowing slight pressurization from volatilization of the ethanol. However, as the temperature within the mill is maintained at approximately 60 to 70 degrees Celsius and the boiling point of the ethanol cover is 77 degrees Celsius, the internal mill pressure does not exceed 200 kPa. The mill is operated at approximately 400 RPM for approximately 20 minutes. The cover may be removed from the mill, and new cover added for a repeat of the milling cycle. While not required, repeated mill cycles with new cover will increase cannabinoid recovery.

The mixture of biomass and cover along with any desired cover is then filtered using a centrifuge to remove the remaining biomass from the cover. The cover is then removed from the cannabinoid essential oils using a vacuum oven. The cover is recovered in a cold trap for reuse. The resulting cannabinoid essential oils are then dissolved in a solvent or solvent mixture suitable to separate the CBD, THC, and other cannabinoids. The separation is performed using a stationary phase column, such as a silica gel column. Once the essential oils are sufficiently separated, the separation solvent also may be removed through vacuum distillation. The resulting essential oils may be used as previously described.

To provide a clear and more consistent understanding of the specification and claims of this application, the following definitions are provided.

Carotenoids, also called tetraterpenoids, are organic pigments produced by plants and algae, as well as several bacteria and fungi.

Oleoresins are semi-solid extracts composed of a resin in solution in an essential oil, which is conventionally obtained by evaporation of the solvent(s) used for their production. In contrast to hydrophilic essential oils often obtained by steam distillation, oleoresins abound in heavier, less volatile and lipophilic compounds, such as resins, waxes, fats and fatty oils.

A solution, in comparison to a suspension, is a liquid where the solvent and solute are homogeneously combined to form a single phase and the solid or liquid solute is dissolved in the solvent. There is no discernable space between the molecules of the solute and the solvent, and once dissolved, the solute will not settle from the solvent without a volume, temperature, or pressure change.

A suspension is a liquid where the liquid and solid particulates are heterogeneously mixed and space exists between the solid particulates and the liquid. The particulates will eventually settle from the liquid, unless the suspension is a colloid, where the particulates are too small to settle.

While various aspects of the invention are described, it will be apparent to those of ordinary skill in the art that other embodiments and implementations are possible within the scope of the invention. Accordingly, the invention is not to be restricted except in light of the attached claims and their equivalents.

1. A method of extracting and isolating an essential oil from a biomass, the method comprising:
combining a biomass including an essential oil with a cover within an attrition mill, where
the essential oil is soluble in the cover, and
the attrition mill includes milling media;
milling the biomass and the cover in the attrition mill for a duration;
reducing the particulate size of the biomass during the milling by repeatedly contacting the biomass with the milling media;
releasing the essential oil from the biomass to the cover during the milling;
dissolving at least a portion of the essential oil released from the biomass in the cover during the milling, where
the cover reduces the oxidation of the released essential oil in relation to the milling without the cover;
forming a mixture during the milling including
a solution of the essential oil in a solvent, where the essential oil is a solute and the cover includes the solvent, and
a milled byproduct biomass; and
separating the solution from the milled byproduct biomass, where the solution includes the essential oil.

2. The method of claim 1, further comprising reducing the particulate size of the essential oil by repeatedly contacting the released essential oil with the milling media.

3. The method of claim 2, where the milling duration continues until the average particulate diameter of the essential oil is from 100 nanometers to 100 microns.

4. The method of claim 2, where the milling duration continues until the average particulate diameter of the essential oil is from 100 nanometers to 30 microns.

5. The method of claim 1, the mill including interior surfaces, the interior surfaces and the milling media substantially non-reactive to the essential oil and the cover.

6. The method of claim 1, where the essential oil is selected from the group consisting of astaxanthin, sea food extract, collagen extract, docosahexaenoic acid, eicosapentaenoic acid, capsaicin, dihydrocapsaicin, cannabinoids, lycopene, hop concentrate, germ oil, ginsenoside, oil of grape seed, lecithin, and pigment.

7. The method of claim 1, where the biomass comprises a material selected from the group consisting of Haematococcus pluvialis algae, peppers, cannabis, garlic, tomatoes, hops, wheat, ginseng, and grapes.

8. The method of claim 1, where the biomass comprises a material selected from the group consisting of green shell mussels, shark cartilage, shell fish, fish, collagen, and egg yolk.

9. The method of claim 1, where the cover is selected from the group consisting of olive oil, sunflower oil, fish oil, vegetable oil, ethanol, an oxidatively inert gas under the milling conditions, and combinations thereof.

10. The method of claim 1, where the biomass comprises Haematococcus pluvialis algae and the essential oil comprises astaxanthin.

11. The method of claim 10, where the cover comprises ethanol.

12. The method of claim 10, where the cover consists essentially of ethanol.

13. The method of claim 10, further comprising maintaining the temperature within the mill below room temperature during a majority of the milling duration.

14. The method of claim 1, where the biomass comprises habanero pepper fruit and the essential oil comprises capsaicin compounds.

15. The method of claim 14, where the cover comprises ethanol.

16. The method of claim 14, where the cover consists essentially of ethanol.

17. The method of claim 14, further comprising maintaining the temperature within the mill above 60 degrees Celsius during a majority of the milling duration.

18. The method of claim 1, where the biomass comprises cannabis plant structures and the essential oil comprises cannabinoids.

19. The method of claim 18, where the cover comprises ethanol.

20. The method of claim 18, where the cover consists essentially of ethanol.

21. The method of claim 18, further comprising maintaining the temperature within the mill above 60 degrees Celsius during a majority of the milling duration.

22. The method of claim 1, further comprising removing the mixture from the mill after the milling and before the separating.

23. The method of claim 22, where the removing the mixture from the mill includes rinsing the interior surfaces of the mill with additional cover.

24. The method of claim 1, where the milling continues until aggregated particulates of the essential oil reach an average diameter of less than 3 microns.

25. The method of claim 1, where the separating the solution from the milled byproduct biomass includes settling the milled byproduct biomass from the solution.

26. The method of claim 25, where the settling includes subjecting the mixture to centrifugal force.

27. The method of claim 1, further comprising at least partially evaporating the solvent from the solution.

28. The method of claim 27, further comprising dosing the essential oil into a carrier oil.

29. The method of claim 27, further comprising forming the essential oil into a powder.

30. The method of claim 1, further comprising drying and shredding the biomass before combining the biomass with the cover.

31. The method of claim 1, further comprising:
mixing the essential oil with a liquefied gelling agent;
forming the essential oil and the liquefied gelling agent mixture into one or more desired portions and shapes; and
allowing the gelling agent to set.

32. The method of claim 31, the mixing further comprising mixing at least one additional nutrient with the liquefied gelling agent.

33. The method of claim 32, the at least one additional nutrient comprising at least one of eicosapentaenoic acid and docosahexaenoic acid.

34. The method of claim 32, the mixing further comprising mixing at least one additional ingredient with the liquefied gelling agent, the additional ingredient selected from the group consisting of a flavoring ingredient, a coloring ingredient, a preservative, and combinations thereof.

35. A method of producing a solution of astaxanthin from a Haematococcus pluvialis algae biomass, the method comprising:
combining an initial feedstock of healthy algae and nutrients in water;
amplifying the algae concentration in the water during a growth phase, the growth phase comprising
supplying light from a light source and carbon dioxide to the initial feedstock, and
supplying the nutrients in the water;
removing at least a portion of the nutrients from the water after the growth phase;
stressing the amplified algae by supplying additional light and carbon dioxide to the amplified algae to promote cyst formation by the amplified algae;
combining the amplified algae with ethanol in an attrition mill including milling media;
milling the amplified algae in the ethanol in the attrition mill to release astaxanthin to the ethanol;
reducing oxidation of the released astaxanthin with the ethanol;
dissolving at least a portion of the astaxanthin in the ethanol to form a mixture comprising
a solution of astaxanthin in the ethanol, where the astaxanthin is a solute and the ethanol is a solvent, and
a milled byproduct of the amplified and stressed algae; and
separating the solution from the milled byproduct of the amplified algae.

36.-48. (canceled)

49. A method of manufacturing a bioavailable essential oil enriched food additive or feed, the method comprising:
combining a biomass including an essential oil with a first cover within a mill, where
the essential oil is soluble in the first cover;
milling the biomass and the first cover in the mill to release the essential oil to the first cover, where
the first cover reduces oxidation of the essential oil, and
at least a portion of the essential oil dissolves in the first cover to produce a mixture comprising
a solution of the essential oil in the first cover, where the essential oil is a solute and the first cover includes a solvent, and
a milled byproduct biomass;
separating the solution from the milled byproduct biomass;
blending the solution with an edible material;
milling the solution and the edible material to transfer at least a portion of the essential oil from the solvent to the edible material; and
removing the solvent from the edible material to provide the bioavailable essential oil enriched feed or food additive as the edible material including transferred essential oil.

50.-58. (canceled)

59. A method of manufacturing a bioavailable essential oil enriched food additive or feed including biomass originating the essential oil, the method comprising:
combining a biomass including an essential oil with a cover within a mill, where
the essential oil is soluble in the cover;
milling the biomass and the cover in the mill to release the essential oil to the cover, where
the cover reduces oxidation of the essential oil, and
at least a portion of the essential oil dissolves in the cover to produce a mixture comprising
a solution of the essential oil in the cover, where the essential oil is a solute and the cover includes a solvent, and
a milled byproduct biomass;
washing the milled byproduct biomass with additional solvent for the essential oil to provide additional solution including the essential oil; and
separating the solution and the additional solution from the milled byproduct biomass to provide the bioavailable essential oil enriched food additive or feed as the milled byproduct biomass.

60.-66. (canceled)
